(19) United States
(12) Patent Application Publication
(10) Pub. No.: US 2010/0184998 A1
(43) Pub. Date: Jul. 22, 2010

(54) ORGANIC COMPOUNDS

(76) Inventors: Beatriz Dominguez, Suffolk (GB); Alan Dyke, Southampton (GB); William Hems, Norwich (GB); Christian Mathes, Offenburg (DE); Anthony C. O'Sullivan, Basel (CH); Gottfried Sedelmeier, Schallstadt (DE)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 12/663,998

(22) PCT Filed: Jun. 18, 2008
(86) PCT No.: PCT/EP2008/057655

§ 371 Date: Dec. 10, 2009

(30) Foreign Application Priority Data
Jun. 19, 2007 (EP) 07110572.0

(51) Int. Cl. C07C 69/593; C07C 57/13; C07D 413/06; C07C 67/00; C07D 321/00

(52) U.S. Cl. 549/267; 560/190; 562/595; 548/230

(57) ABSTRACT

The present invention relates to olefin metathesis processes for the manufacture of a compound of the formula (I)

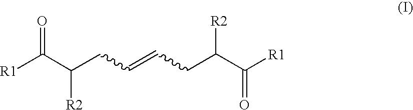

which is a novel useful intermediate in the synthesis of pharmaceutically active compounds.

### FIELD OF THE INVENTION

The invention relates to a novel process, novel process steps and novel intermediates useful in the synthesis of pharmaceutically active compounds, in particular renin inhibitors.

### BACKGROUND OF THE INVENTION

Renin passes from the kidneys into the blood where it affects the cleavage of angiotensinogen, releasing the decapeptide angiotensin I which is then cleaved in the lungs, the kidneys and other organs to form the octapeptide angiotensin II. The octapeptide increases blood pressure both directly by arterial vasoconstriction and indirectly by liberating from the adrenal glands the sodium-ion-retaining hormone aldosterone, accompanied by an increase in extracellular fluid volume which increase can be attributed to the action of angiotensin II. Inhibitors of the enzymatic activity of renin lead to a reduction in the formation of angiotensin I, and consequently a smaller amount of angiotensin II is produced. The reduced concentration of that active peptide hormone is a direct cause of the hypotensive effect of renin inhibitors.

With compounds such as (with INN name) aliskiren {(2S,4S,5S,7S)-5-amino-N-(2-carbamoyl-2-methylpropyl)-4-hydroxy-2-isopropyl-7-[4-methoxy-3-(3-methoxypropoxy)benzyl]-8-methylnonanamide}, a new antihypertensive has been developed which interferes with the renin-angiotensin system at the beginning of angiotensin II biosynthesis.

As the compound comprises 4 chiral carbon atoms, the synthesis of the enantiomerically pure compound is quite demanding. Therefore, amended routes of synthesis that allow for more convenient synthesis of this sophisticated type of molecules are welcome.

It is therefore a problem to be solved by the present invention to provide new synthesis routes and new intermediates allowing a convenient and efficient access to this class of compounds. The present invention relates thus to a process for the manufacture of useful intermediate in the synthesis of pharmaceutically active compounds, in particular renin inhibitors, such as renin inhibitors comprising a 2,7-dialkyl-4-hydroxy-5-amino-8-aryl-octanoyl amide backbone, such as aliskiren or pharmaceutically acceptable salts thereof.

### SUMMARY OF THE INVENTION

During an investigation into the preparation of alternative intermediates towards the total synthesis of renin inhibitors, in particular renin inhibitors comprising a 2,7-dialkyl-4-hydroxy-5-amino-8-aryl-octanoyl amide backbone, a C-8 molecule characterized by the presence of an “inner” double bond and two chiral centers was identified as a key substrate. The synthesis of this 4-octen-1,8-dioic acid molecule, of general formula (I), is undertaken following olefin metathesis strategies, wherein the key metathesis reaction employs, for example, a ruthenium metal carbene complex as described herein.

Said strategies have thus as a key common feature the assemblage of the C-8 octa-1,8-dioic acid scaffold of the compound of formula (I) via an olefin metathesis reaction step. Both intra-molecular and inter-molecular olefin metathesis processes can be used to assembly such a C-8 scaffold, which is then further elaborated into the 4-octen-1,8-dioic acid molecule of formula (I). The invention is thus directed to olefin metathesis methods for preparing a compound of formula (I), in particular, wherein the C-8 scaffold of a compound of formula (I) is either build via cross-metathesis (inter-molecular olefin methathesis) or via ring-closing metathesis (intra-molecular olefin metathesis) reactions.

In one of these olefin metathesis strategies, the C-8 scaffold of a compound of formula (I) is built as a triene, of general formula (III), by cross-metathesis reaction of a C-5 diene compound of general formula (II). The chiral centers are then introduced by asymmetric reduction of the “outer” double bonds by the use of a chiral hydrogenation catalyst to yield the compound of formula (I). The intra-molecular olefin metathesis variant of this approach is also possible. In said variant the C-8 octa-1,8-dioic acid scaffold of the compound of formula (I) is build by ring-closing metathesis of the linked bis-C-5 diene compound of general formula (IIa). Further hydrogenation and hydrolysis steps lead to the compound of formula (I).

In another olefin metathesis strategy, a cross-metathesis reaction of an alternative C₁₋₅ compound, of general formula (IV) is the key step for the synthesis of the C-8 scaffold of a compound of formula (I). The intra-molecular olefin metathesis variant of this approach is also possible. In said variant the C-8 octa-1,8-dioic acid scaffold of the compound of formula (I) is build by ring-closing metathesis of the linked bis-C-5 diene compound of general formula (IVa). A later hydrolysis step leads to the compound of formula (I).

In a further embodiment, the invention relates to products obtainable by any of the processes, described herein, en route to the compound of general formula (I), and to their use in the production of renin inhibitors, in particular renin inhibitors comprising a 2,7-dialkyl-4-hydroxy-5-amino-8-aryl-octanoyl amide backbone. Moreover, any of the process steps of the present invention either alone or in a suitable combination may be employed in the synthesis of a renin inhibitor, in particular renin inhibitors comprising a 2,7-dialkyl-4-hydroxy-5-amino-8-aryl-octanoyl amide backbone, such as aliskiren or a pharmaceutically acceptable salt thereof.

### DETAILED DESCRIPTION OF THE INVENTION

In a first aspect, the invention relates to a process for the manufacture of a compound of the formula (I)

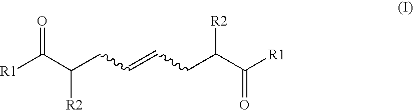

wherein

### R1 is OR3 or NR4R5;

R2 is C₁₋₇alkyl or C₃₋₈cycloalkyl;
R3 is hydrogen, C₁₋₄alkyl, phenyl- or naphthyl-C₁₋₄alkyl, aryl or C₃₋₈cycloalkyl, each unsubstituted or substituted; or is SiRR′R″, wherein R, R′ and R″ are independently of each other C₁₋₇alkyl, aryl or phenyl-C₁₋₄alkyl;
R4 and R5 are independently hydrogen, C₁₋₇alkyl, phenyl- or naphthyl-C₁₋₄alkyl, aryl or C₃₋₈cycloalkyl, each unsubstituted or substituted;
or R4 and R5 may form together a 3 to 7 membered nitrogen containing saturated hydrocarbon ring, which may contain one or more heteroatoms selected from N or O and, which can be unsubstituted or substituted;
or a salt thereof;
said process comprising one or more of the following steps:
a) subjecting a compound of formula (II), or a salt thereof,

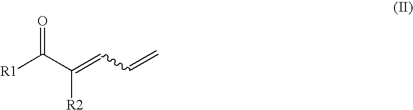

wherein R1 and R2 are as defined for a compound of formula (I), to cross-metathesis reaction to obtain a compound of formula (III), or a salt thereof,

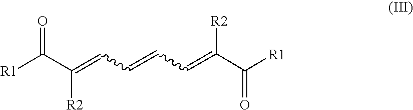

wherein R1 and R2 are as defined for a compound of formula (I);

b) subjecting said compound of formula (III), or a salt thereof, to hydrogenation to obtain a compound of formula (I), or a salt thereof.

In a further aspect, the present invention is related to compounds of formula (I)

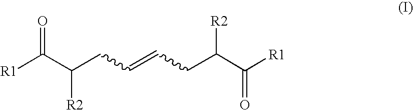

wherein

### R1 is OR3 or NR4R5;

R2 is C₁₋₇alkyl or C₃₋₈cycloalkyl;
R3 is hydrogen, C₁₋₇alkyl, phenyl- or naphthyl-C₁₋₄alkyl, aryl or C₃₋₈cycloalkyl, each unsubstituted or substituted; or is SiRR′R″, wherein R, R′ and R″ are independently of each other C₁₋₇alkyl, aryl or phenyl-C₁₋₄alkyl;
R4 and R5 are independently hydrogen, C₁₋₇alkyl, phenyl- or naphthyl-C₁₋₄alkyl, aryl or C₃₋₈cycloalkyl, each unsubstituted or substituted;
or R4 and R5 may form together a 3 to 7 membered nitrogen containing saturated hydrocarbon ring, which may contain one or more heteroatoms selected from N or O and, which can be unsubstituted or substituted;
or a salt thereof.

In one embodiment, R2 is straight chain or branched, in particular branched, C₁₋₇alkyl, such as C₁₋₄ alkyl, for example methyl, ethyl or isopropyl, in particular isopropyl.

In another embodiment, R1 is OR3, wherein R3 is for example hydrogen or C₁₋₇alkyl; in particular hydrogen, methyl or ethyl. In one embodiment R1 is for example OH.

In yet another embodiment, R1 is NR4R5, wherein R4 and R5 are straight chain or branched C₁₋₇alkyl, such as n-butyl or isopropyl, in particular isopropyl. In yet another embodiment R4 and R5 may form together a, substituted or unsubstituted, 3 to 7 membered nitrogen containing saturated hydrocarbon ring, which may contain one or more heteroatoms selected from N or O, such as a 1,3-oxazolidin-2-onyl ring.

In one embodiment, the compound according to formula (I), or a salt thereof, has the following stereochemistry

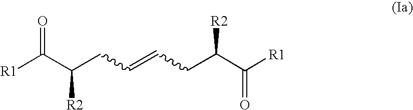

wherein R1 and R2 are as defined for a compound of formula (I), in particular as defined in those embodiments mentioned earlier for a compound of formula (I).

In another embodiment, a compound according to formula (I), or a salt thereof, has the following stereochemistry

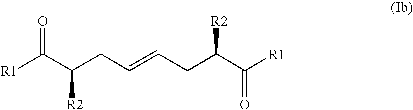

wherein R1 and R2 are as defined for a compound of formula (I), in particular wherein R1 is OH and R2 is a branched C₁₋₇ alkyl, such as isopropyl.

All these compounds are key intermediates in the synthesis of renin inhibitors, in particular renin inhibitors comprising a 2,7-dialkyl-4-hydroxy-5-amino-8-aryl-octanoyl amide backbone, such as aliskiren or any pharmaceutical salt thereof.

In another aspect, the subject-matter of the present invention is also directed to compounds of formula (III), or salts thereof,

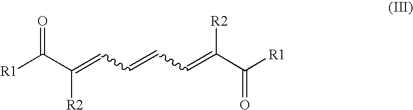

wherein R1 and R2 are as defined for a compound of formula (I), in particular as described in those embodiments mentioned earlier for a compound of formula (I).

In one embodiment, the compound according to formula (III), or a salt thereof, has the following structure:

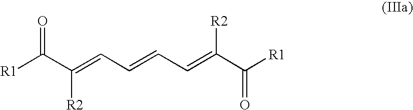

wherein R1 and R2 are as defined for a compound of formula (I), in particular compounds of formula (IIIa), or salts thereof, wherein R1 is OH and R2 is a branched C₁₋₇alkyl, such isopropyl. In another embodiment, compounds of formula (III) are compounds of formula (IIIa), or salts thereof, wherein R1 is NR4R5, in particular wherein R4 and R5 are isopropyl. In yet another embodiment R4 and R5 may form together a, substituted or unsubstituted, 3 to 7 membered nitrogen containing saturated hydrocarbon ring, which may contain one or more heteroatoms selected from N or O, such as piperidine or oxazolidinone.

Therefore, in a very relevant aspect, this invention relates to a process for the manufacture of a compound of the formula (III), or a salt thereof,

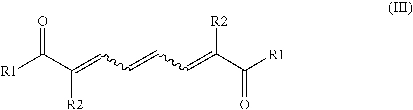

wherein R1 and R2 are as defined above, said process comprising the step of subjecting a compound of formula (II), or a salt thereof,

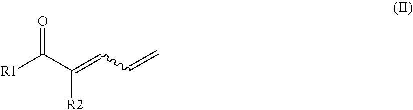

wherein R1 and R2 are as defined for a compound of formula (I), to cross-metathesis reaction to obtain a compound of formula (III), or a salt thereof.

Starting compounds of formula (II), or salts thereof, can be easily obtained by an aldol condensation approach as shown in Scheme 1.

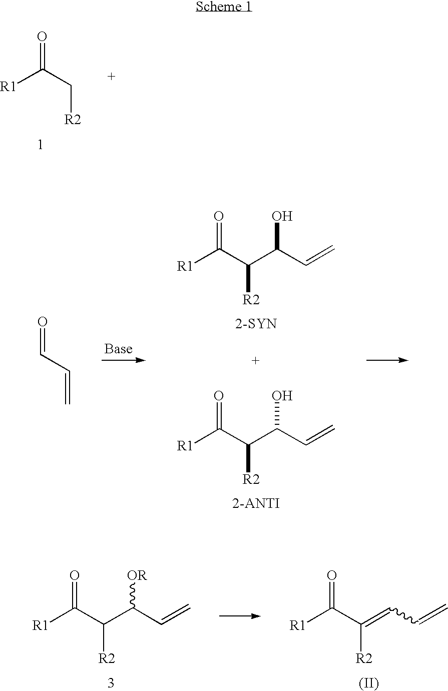

Reaction of the enolate of ketone 1, which can be prepared by the use of a base such as lithium diisopropylamide, lithium hexamethyldisilazide, sodium hexamethyldisilazide, potassium hexamethyldisilazide or lithium 2,2,6,6-tetramethylpiperidide, with acroleine gives the corresponding 2-syn and 2-anti aldol adducts. Conversion of the hydroxyl group into a good leaving group, for example by mesylation or tosylation, according to standard methods, followed by elimination upon reaction with a base, such as NaOMe, KOMe, LiOMe or KO^{t}Bu, affords compounds of formula (II). In one particular embodiment, a compound of formula (II), wherein R1=OEt and R2=ⁱPr, can be prepared by following said sequence. The elimination of the corresponding mesylate intermediate with 2 equivalents of NaOMe at room temperature overnight can provide said ester of formula (II) in e.g. a 20:1 E/Z ratio.

The process step of cross-metathesis reaction of compounds of formula (II), or salts thereof, is carried out with or without an added solvent, in one embodiment it is carried out with solvent. Examples of solvents include hydrocarbons such as hexane, heptane, benzene, toluene and xylene; chlorinated hydrocarbons such as dichloromethane, dichloroethane, chlorobenzene and dichlorobenzene; ethers such as diethyl ether, diisopropyl ether, tetrahydrofuran, and methyl tert-butyl ether; and esters such as ethyl acetate, n-propyl acetate, and methyl butyrate. Further examples of solvents are toluene, dichloromethane or dichloroethane, in one embodiment the solvent is dichloromethane. Solvents are in particular degassed according to standard techniques well known in the art. The amount of solvent employed may be in the range of zero to 150 mL per mmol of reactant (II), for example in the range of 1 to 100 mL per mmol of reactant (II), such as in the range of 1 to 50 mL per mmol of reactant (II), in particular in the range of 1 to 10 mL per mmol of reactant (II). The reaction is in particular conducted under inert atmosphere. The term “inert” as used throughout this application, means unreactive with any of the reactants, solvents, or other components of the reaction mixture. Such inert conditions are generally accomplished by using inert gas such as carbon dioxide, helium, nitrogen, argon, among other gases. This process step of is typically carried out at a temperature in the range of from −10 to 150° C., for example at a temperature in the range of from 0 to 100° C., such as at a temperature in the range of from 20 to 80° C., in particular at a temperature in the range of from 40 to 80° C.

As described in US Patent Application No 20060030742; metathesis catalyst for cross-metathesis may be any heterogeneous or homogeneous transition metal compound which is effective for catalyzing metathesis reactions and is compatible with the functional groups present in the reactants. In particular metathesis catalysts are heterogeneous or homogeneous compounds of transition metals selected from Groups 4 (IVA) and 6-10 (VIA-10) of the Periodic Table of the Elements. By the term “heterogeneous compound” it is meant any transition metal or metal compound of Groups 4 and 6-10 of the Periodic Table of the Elements admixed with, supported on, ion-exchanged with, deposited on, or co-precipitated with common inert support materials such as silica, alumina, silica-alumina, titania, zirconia, carbon, and the like. The support material also may be a acidic or basic macroreticulated ion-exchange resin. The term “homogeneous compound” means any Group 4 or Group 6-10 transition metal compound that is soluble or partly soluble in the reaction mixture. Effective metathesis catalysts may be prepared by methods well known to practitioners skilled in the art and are described in chemical journals such as Mol et al Catal. Today, 1999, 51, 289-99 and in PCT Application No. 02/00590; European Application No. 1 022 282 A2; and U.S. Pat. Nos. 5,922,863; 5,831,108; and 4,727,215. For the present cross-metathesis of compounds of formula (II), or salts thereof, the olefin metathesis catalyst is, for example, a ruthenium alkylidene catalyst, in particular ruthenium alkylidene catalysts such as:

| | |
|---|---|
| 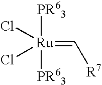 | 1a, R⁶ = Cyclohexenyl, R⁷ = Ph 1b, R⁶ = Cyclohexenyl, R⁷ = CH₂Ph 1c, R⁶ = ⁱPr, R⁷ = C₅H₁₁ 1d, R⁶ = ⁱPr, R⁷ = C₇H₁₅ 1e, R⁶ = ⁱPr, R⁷ = CH₂Ph 1f, R⁶ = ⁱPr, R⁷ = CH₂SPh 1g, R⁶ = ⁱPr, R⁷ = CHCPh₂ |
| 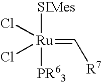 | 2a, R⁶ = Cyclohexenyl, R⁷ = Ph 2b, R⁶ = ⁱPr, R⁷ = CH₂Ph 2c, R⁶ = ⁱPr, R⁷ = CH₂SPh 2d, R⁶ = Ph, R⁷ = CH₂Ph 2e, R⁶ = Tol, R⁷ = CH₂Ph 2f, R⁶ = p-MeOC₆H₄, R⁷ = CH₂Ph 2g, R⁶ = C₇H₁₅, R⁷ = ⁱPr |
| 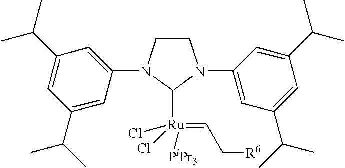 | 3a, R⁶ = C₄H₉ 3b, R⁶ = C₆H₁₃ 3c, R⁶ = Ph |
| 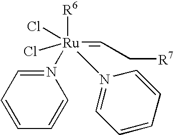 | 4a, R⁶ = IMes, R⁷ = Ph 4b, R⁶ = SIMes, R⁷ = Ph 4c, R⁶ = SIMes, R⁷ = C₆H₁₃ |
| 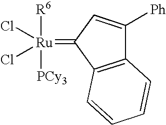 | 5a, R⁶ = SIMes 5b, R⁶ = P(Cyclohexenyl)₃ |
| 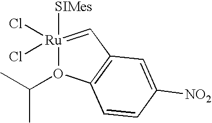 | 6a |
| 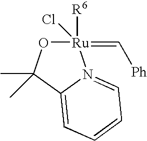 | 7a, R⁶ = P(Cyclohexenyl)₃ 7b, R⁶ = SIMes 7c, R⁶ = P(ⁱPr)₃ |
| 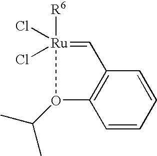 | 8a, R⁶ = P(Cyclohexenyl)₃ 8b, R⁶ = SIMes |
| 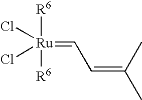 | 9a, R⁶ = P(Cyclohexenyl)₃ |
| 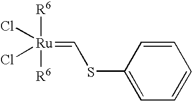 | 10a, R⁶ = P(Cyclohexenyl)₃ |

wherein the terms IMes and SIMes represent N,N′-bis(mesityl)imidazol-2-ylidene and 3-bis(mesityl)imidazolidene-2-ylidene ligands, respectively; and wherein the terms ⁱPr, Ph and Tol mean isopropyl, phenyl and tolyl.

Catalyst 1a (Grubbs' first-generation) is available from Sigma-Aldrich. The preparation and use of first-generation Grubbs' catalyst are described in chemical journals such as: Schwab, P.; France, M. B.; Ziller, J. W.; Grubbs, R. H. Angew. Chem. Int. Ed. Engl. 1995, 34, 2039; Schwab, P.; Grubbs, R. H.; Ziller, J. W. J. Am. Chem. Soc. 1996, 118, 100 and Welheim, T. E.; Belderrain, T. R.; Brown, S, N.; Grubbs, R. H. Organometallics 1997, 16, 3867. Catalyst 2a (Grubbs' second-generation) is available from Sigma-Aldrich. The preparation and use of second-generation Grubbs' catalyst are described in chemical journals such as: Scholl, M.; Ding, S. C.; Lee, W.; Grubbs, R. H. Org. Lett. 1999, 1, 953; Bielawski, C. W.; Grubbs, R. H. Angew. Chem., Int. Ed. 2000, 39, 2903; Trnka, T. M.; Morgan, J. P.; Sanford, M. S.; Wilhelm, T. E.; Scholl, M.; Choi, T.-L.; Ding, S.; Day, M. W.; Grubbs, R. H. J. Am. Chem. Soc. 2003, 125, 2546 and Love, J. A.; Sanford, M. S.; Day, M. W.; Grubbs, R. H. J. Am. Chem. Soc. 2003, 125, 10103. The preparation of catalysts 1b-g, 2b-g and 3a-c is described in U.S. Pat. No. 5,912,376. The preparation and use of catalysts 4a-c (Grubbs' third-generation) are described in chemical journals such as: Sanford, M. S.; Love, J. A.; Grubbs, R. H. Organometallics 2001, 20, 5314 and Love, J. A.; Morgan, J. P.; Trnka, T. M.; Grubbs, R. H. Angew. Chem., Int. Ed. 2002, 41, 4035. Catalysts 5a,b are available from Strem Chemicals. And their preparation and use are described in chemical journals such as: Jafarpour, L.; Schanz, H.-J.; Stevens, E. D.; Nolan, S. P. Organometallics 1999, 18, 5416; Fürstner, A.; Thiel, O. R.; Ackermann, L.; Nolan, S. P.; Schanz, H.-J. J. Org. Chem. 2000, 65, 2204; Fürstner, A; Guth, O; Düffels, A.; Seidel, G.; Liebl, M.; Gabor, B.; Mynott, R. Chem. Eur. J. 2001, 7, 4811; Fürstner, A.; Schlede, M. Adv. Synth. Catal. 2002, 344, 657 and Opstal, T.; Verpoort, F. New J. Chem. 2003, 27, 257.

The preparation and use of catalyst 6a are described in chemical journals such as: Grela, K.; Harutyunyan, S.; Michrowska, A. Angew. Chem., Int. Ed. 2002, 41, 4038; Michrowska, A.; Bujok, R.; Harutyunyan, S.; Sashuk, V.; Dolgonos, G.; Grela, K. J. Am. Chem. Soc. 2004, 126, 9318 and Harutyunyan, S.; Michrowska, A.; Grela, K. in Catalysts for Fine Chemical Synthesis; Roberts, S. M., Whittall, J., Mather, P., McCormack, P., Eds.; Wiley-Interscience: New York 2004; Vol. 3, 169. The preparation of catalysts 7a-c is described in chemical journals such as: Van der Schaaf, P. A.; Mühlebach, A.; Hafner, A.; Kolly, R. Catalysts 8a (Hoveyda-Grubbs' first-generation) and 8b (Hoveyda-Grubbs' second-generation) are available from Sigma-Aldrich and, their preparation and use are described in chemical journals such as: Kingsbury, J. S.; Harrity, J. P. A.; Bonitatebus, P. J.; Hoveyda, A. H. J. Am. Chem. Soc. 1999, 121, 791; Garber, S. B.; Kigsbury, J. S.; Gray, B. L.; Hoveyda, A. H. J. Am. Chem. Soc. 2000, 122, 8168 and Nicola, T.; Brenner, M.; Donsbach, K.; Kreye, P. Org. Proc. Res. Dev. 2005, 9, 513. Catalyst 10a is available from Strem Chemicals and, its preparation and use are described in chemical journals such as: Van der Schaaf, P. A.; Kolly, R.; Kirner, H.-J.; Rime, F.; Mühlebach, A.; Hafner, A. J. Organomet. Chem. 2000, 606, 65 and Katayama, H.; Nagao, M.; Ozawa, F. Organometallics, 2003, 22, 586.

Alternative catalysts are, for example, 11a-e, which are commercially available from Strem or Aldrich.

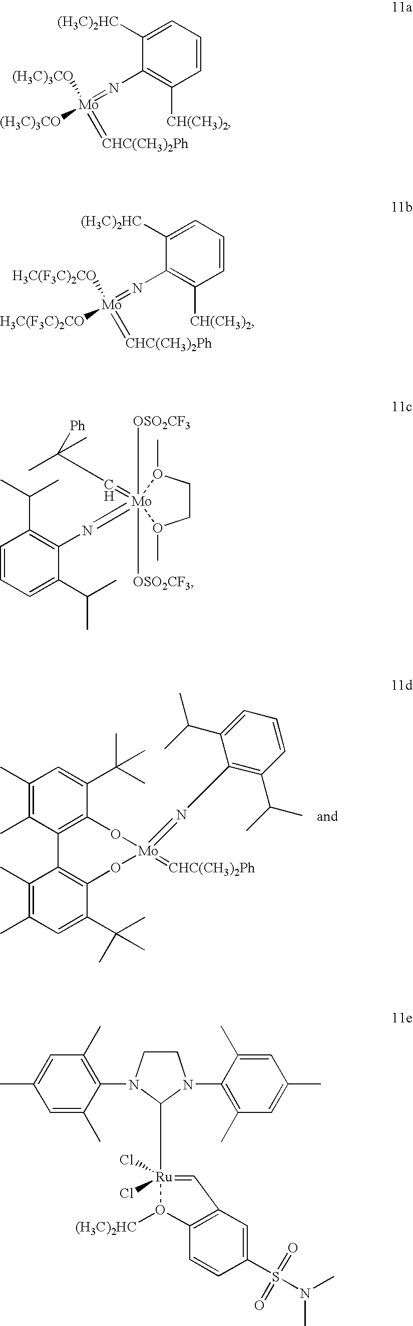

In one embodiment ruthenium alkylidene catalysts are entries 2a (Grubbs' second-generation catalyst), 2g, 4b and 6a; for example 2a and 2g; in particular 2a.

The amount of the metathesis catalyst typically employed in the process may be in the range of from 0.01 (s/c 10000/1) to 10% mol (s/c 10/1), for example of from 0.05 (s/c 2000/1) to 5% mol (s/c 5/1), such as of from 0.05 (s/c 2000/1) to 1% mol (s/c 100/1), in particular of from 0.05 (s/c 2000/1) to 0.5% mol (s/c 200/1).

It is also possible to influence the properties of the metathesis catalyst employed by the use of specific additives, such as triethylamine, pyridine or AsPh₃.

The cross-metathesis step of the present invention involves single or step-wise addition of the metathesis catalyst. In one particular embodiment, a solution of the catalyst (e.g. 0.05% mol) in CH₂Cl₂ can be added to a diene of formula (II), wherein R1=OEt and R2=ⁱPr, at 30-50° C. in several, such as four, different portions during a period of 1 to 3 hours. Standard conversion can be observed after e.g. 4 hours; by sampling of the reaction mixture at different times after the addition of each catalyst portion, a very fast initial reaction rate can be observed. For the purpose of convenience, single addition of the metathesis catalyst is preferred.

The cross-metathesis reaction is generally complete after a reaction time of from 0.5 to 48 hours. After completion of the reaction, the reaction products of formula (III) may be separated from the reaction mixture by several purification procedures well known to persons skilled in the art including, but not limited to crystallization, distillation, extraction, and the like. For example if the reaction products are volatile, the products may be separated by distillation from the reaction mixture.

In principle the cross-metathesis reaction of a compound of formula (II), or a salt thereof, can provide mixtures of all possible triene stereoisomers (E,E,E/Z,Z,Z/E,E,Z/E,Z,Z/Z,E,Z and E,Z,E) of general formula (III). The E/Z selectivity of the cross-metathesis reaction of the present invention is very high. Thus, in a further embodiment, the present invention provides a process for the stereoselective synthesis of a E,E,E triene of formula (IIIa), or a salt thereof,

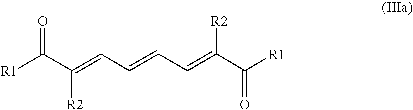

wherein R1 and R² areas defined for a compound of formula (I).

As detailed in Scheme 2, in one embodiment, the cross-metathesis of a 6:1 E/Z mixture of a compound of formula (II), wherein R1=OEt and R2=iPr, provides the corresponding compound of formula (III) in a 67:5:28 E,E,E:E,Z,E:E,E,Z ratio. In another embodiment, a 20:1 E/Z mixture of said compound of formula (I), wherein R1=OEt and R2=iPr, provides the corresponding compound of formula (III) in a 87:5:8 E,E,E:E,Z,E:E,E,Z ratio.

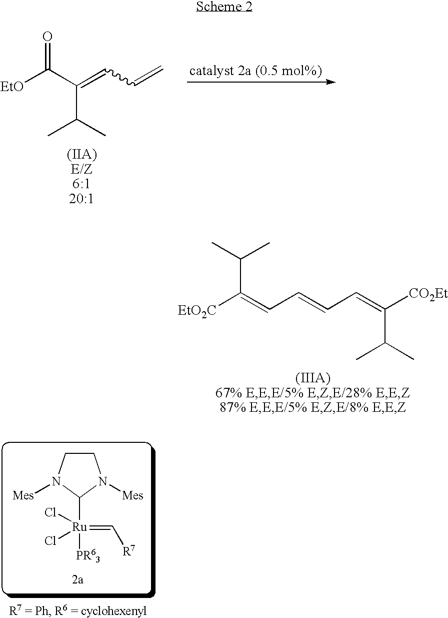

Mixtures of triene isomers of general formula (III) can be subjected to isomerization reaction conditions well known to persons skilled in the art (e.g. Feliu, A. L.; Seltzer, S. J. Org. Chem. 1985, 50, 447). Some of these are exemplified below with respect to specific examples but are generally applicable and are not limited to these examples. Such standard isomerization conditions may provide means to further change the isomeric ratio of compounds of formula (III), or salts thereof, obtained by the use of the process of the present invention. In one embodiment, the cross-metathesis of a E compound of formula (II), wherein R1=NⁱPr₂ and R2=ⁱPr, can provide the corresponding compound of formula (III) in e.g. a 3:1 E,E,E:E,Z,E ratio. Treatment of said resulting mixture of trienes with iodine in hexane can afford a E,E,E:E,Z,E mixture in e.g. a 11:1 ratio, as shown in Scheme 3.

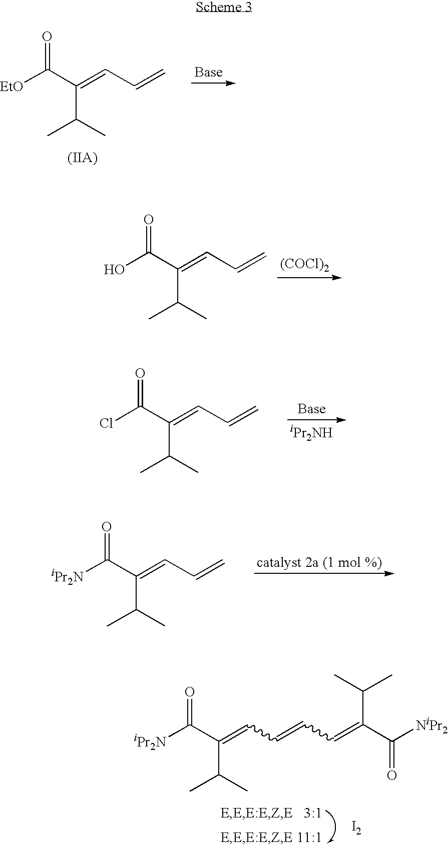

In another embodiment, isomeric mixtures of a compound of formula (III), wherein R1=OEt and R2=ⁱPr, can be also treated with iodine to provide a consistent mixture of (E,E,E)/(E,E,Z)/(Z,E,Z) isomers, e.g. 4:4:1, independently of the composition of the initial mixture (Table 1).

TABLE 1

| Entry | % E, E, E | % E, Z, E | % E, E, Z | % Z, E, Z |
|---|---|---|---|---|
| 1 | 12-38 | 9-2 | 79-47 | 0-13 |
| 2 | 11-39 | 0-0 | 39-45 | 50-16 |
| 3 | 18-40 | 12-2 | 70-46 | 0-12 |
| 4 | 100-40 | 0-0 | 0-45 | 0-15 |

The initial value refers to the initial percentage of a particular diastereoisomer, the second value is the percentage of the diastereoisomer in the mixture after stirring it in I₂/hexane for 24 hours.

In another relevant aspect, the present invention relates to a process for preparing a compound of formula (I), or a salt thereof,

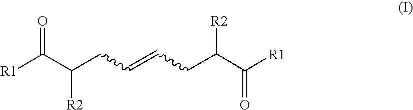

wherein R1 and R2 are as defined above for a compound of formula (I), said process comprising the step of subjecting a compound of formula (III), or a salt thereof,

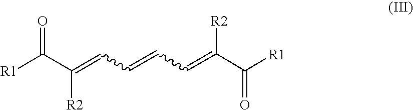

wherein R1 and R2 are as defined for a compound of formula (I), to hydrogenation to obtain a compound of formula (I), or a salt thereof.

Therefore, an embodiment of the process of the present invention comprises the step wherein the compound of formula (III), or a salt thereof, which can be obtained from a compound of formula (II), or a salt thereof, as described earlier, is further reacted to obtain the compound of formula (I), or a salt thereof.

The present invention provides thus a process for hydrogenating a compound of formula (III), or a salt thereof, wherein R1 and R2 are as defined for a compound of formula (I), by bringing said compound into contact with hydrogen in the presence of a catalyst, which comprises as active metal at least one metal of transition group VIII of the Periodic Table (alone or together with at least one metal of transition group I or VIII of the periodic table). In particular, the catalyst comprises for example as active metal rhodium or ruthenium. For the present selective hydrogenation of compounds of formula (III), or salts thereof, the hydrogenation catalyst is, for example, a ruthenium catalyst, in particular a ruthenium catalyst such as:

1-9

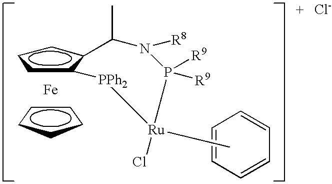

| Catalyst | | |
|---|---|---|
| 1 | [(S)-BoPhoz RuCl benzene)]Cl | R⁸ = Me, R⁹ = Ph |
| 2 | [(S)-BoPhoz RuCl (benzene)]Cl | R⁸ = Me, R⁹ = p-fluorophenyl |
| 3 | [(S)-BoPhoz RuCl (benzene)]Cl | R⁸ = Me, |
| | | R⁹ = 3,5-difluorophenyl |
| 4 | [(R)-BoPhoz RuCl (benzene)]Cl | R⁸ = Me, R⁹ = (R)-binol |
| 5 | [(R)-BoPhoz RuCl (benzene)]Cl | R⁸ = Me, R⁹ = (S)-binol |
| 6 | [(S)-BoPhoz RuCl (benzene)]Cl | R⁸ = Me, R⁹ = p-CF₃phenyl |
| 7 | [(R)-BoPhoz RuCl (benzene)]Cl | R⁸ = Bn, R⁹ = Ph |
| 8 | [(R)-BoPhoz RuCl (benzene)]Cl | R⁸ = (R)-phenethyl, R⁹ = Ph |
| 9 | (S)-BoPhoz RuCl₂ dmf | R⁸ = Me, R⁹ = Ph |

wherein BoPhoz represents a ligand of general formula (V) and binol means 2,2′-dihydroxy-1,1′-dinaphthyl.

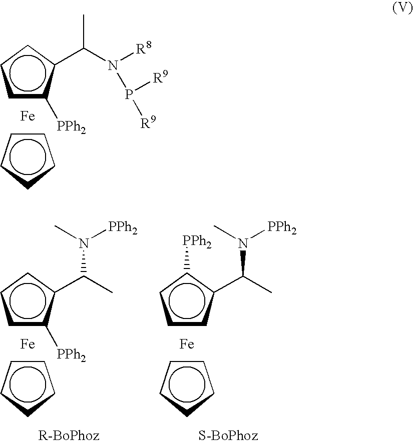

Preparation and use of BoPhoz ligands in Rh complexes is described in: Boaz, N. W.; Debenham, S. D.; Mackenzie, E. B.; Large, S. E. Org. Lett. 2002, 4, 2421; Boaz, N. W.; Debenham, S. D.; Large, S. E.; Moore, M. K. Tetrahedron: Asymmetry 2003, 14, 3575; Jia, X.; Li, X.; Lam, W. S.; Kok, S. H. L.; Xu, L.; Lu, G.; Yeung, C.-H.; Chan, A. S.C. Tetrahedron: Asymmetry 2004, 15, 2273 and Boaz, N. W.; Large, S. E.; Ponasik, J. A., Jr.; Moore, M. K.; Barnette, T.; Nottingham, W. D. Org. Process Res. Dev. 2005, 9, 472.

The use of ruthenium complexes of BoPhoz ligands for the asymmetric hydrogenation of functionalized ketones has been recently described in: Boaz, N. W.; Ponasik, J. A., Jr.; Large, S. E. Tetrahedron Lett. 2006, 47, 4033.

In one embodiment, the hydrogenation catalyst used in the present invention is selected from the group of: [(S)-p-fluorophenylMeBoPhoz RuCl (benzene)]Cl (2), [(S)-3,5-difluorophenylMeBoPhoz RuCl (benzene)]Cl (3), [(S)-p-CF₃-phenylMeBoPhoz RuCl (benzene)]Cl (6), [(R)-BnBoPhoz RuCl (benzene)]Cl (7) and [(R)-phenethyl-(R)-BoPhoz RuCl (benzene)]Cl (8); in particular [(S)-3,5-difluorophenylMeBoPhoz RuCl (benzene)]Cl (3) and [(R)-phenethyl-(R)-BoPhoz RuCl (benzene)]Cl (8).

The amount of catalyst typically employed in the process may be in the range of from 0.01 to 10% mol, in one embodiment of from 0.05 to 5% mol, in another embodiment of from 0.05 to 2% mol, in yet another embodiment of from 0.05 to 1% mol.

The hydrogenation may be carried out at a hydrogen pressure in the range of from 1 to 400 bars, in one embodiment of from 1 to 300 bars, in another embodiment of from 10 to 150 bars. In one embodiment, reaction temperature is in the range of from 20 to 200° C., in another embodiment of from 20 to 100° C. and in a further embodiment of from 20 to 80° C.

It is also possible to influence the properties of the hydrogenation catalyst employed by the use of specific additives, such as triethylamine, sodium methoxide or fluoroboric acid.

The hydrogenation reaction is generally complete after a reaction time of from 1 to 48 hours. After completion of the reaction, the reaction products may be separated from the reaction mixture by several purification procedures well known to persons skilled in the art, as mentioned earlier.

In another relevant aspect, the present invention relates to a process for preparing a compound of formula (I), or a salt thereof,

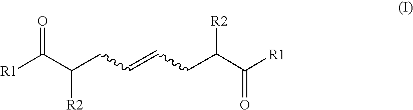

wherein R1 and R2 are as defined above for a compound of formula (I), said process comprising the step of subjecting a compound of formula (IIIa), or a salt thereof,

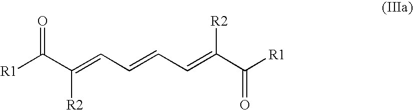

wherein R1 and R2 are as defined for a compound of formula (I), to hydrogenation to obtain a compound of formula (I), or a salt thereof.

In one embodiment, the hydrogenation reaction of a compound of formula (IIIa), or a salt thereof, takes place under the same conditions mentioned above for compounds of formula (III).

In one embodiment, the present invention provides a process for hydrogenating a compound of formula (IIIa), wherein R1=OH and R2=isopropyl. Said novel dicarboxylic acid, which is also an embodiment of the present invention, may be obtained by hydrolysis reaction of the triene ester of formula (IIIa), wherein R1=OEt and R2=isopropyl, according to methods well known in the art and as described herein. Said triene ester may be obtained from the cross-metathesis reaction detailed above. Specifically, the (E,E,E)-triene of formula (IIIa), wherein R1=OR3 such as OEt and R2=isopropyl, can be converted into the corresponding (E,E,E)-bisacid under basic hydrolysis conditions. In particular, said triene ester can be dissolved in e.g. a 1:1 mixture of THF/MeOH, treated with a base such as 2M LiOH and stirred over night at 60-100° C., such as 80° C., to give said (E,E,E)-bisacid (Scheme 4).

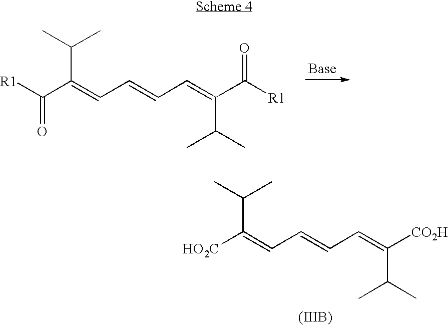

The diastereoselctivity of the hydrogenation reaction of compounds of general formulae (III) and (IIIa) is high. In one embodiment, the hydrogenation reaction of the compound of formula (IIIa) wherein R1=OH and R2=isopropyl can provide the corresponding compound of formula (I) in e.g. 7:1 dl:meso. The separation of (IB)-D,L and (IB)-meso cab be achieved, for example, via recrystallization of diastereomeric salts by several procedures well known to persons skilled in the art (e.g. Kozma, D. CRC Handbook of Optical Resolutions via Diastereomeric Salt Formation, CRC Press, 2002).

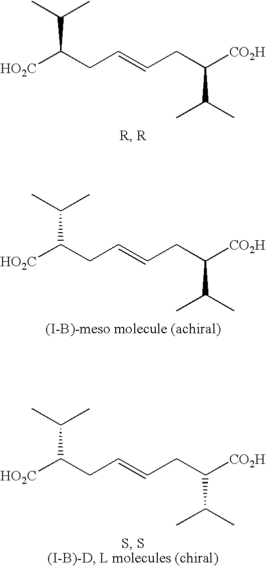

Accordingly, the present invention provides a process in which a triene compound of formula (III), or a salt thereof, is hydrogenated in a chemoselective and diastereoselective manner in the presence of an olefin hydrogenating catalyst to provide a compound of formula (I), or a salt thereof, in particular a compound of formula (Ia), or a salt thereof, or a compound of formula (Ib); or a salt thereof, wherein R1 and R2 are as defined earlier, in particular wherein R1 and R2 substituents are as mentioned in earlier embodiments.

In general, the selective hydrogenation of compounds containing multiple bonds is challenging. The desired product may be obtained, if at all, along with undesired more highly or completely saturated products.

Processes for the selective hydrogenation of α,β-unsaturated acids are reported in the literature. The asymmetric hydrogenation of several α,β-unsaturated carboxylic acids by the use of BINAP-Ru(II) dicarboxylate complexes is described in chemical journals such as: Noyori, R.; Ohta, M.; Hsiao, Y.; Kitamura, M.; Ohta, T.; Takaya, H. J. Am. Chem. Soc. 1986, 108, 7117; Ohta, T.; Takaya, H.; Kitamura, M.; Nagai, K.; Noyori, R. J. Org. Chem. 1987, 52, 3174; Ohta, T.; Takaya, H.; Noyori, R. Inorg. Chem. 1988, 27, 566; Ohta, T.; Takaya, H.; Noyori, R. Tetrahedron Lett. 1990, 31, 7189; Ashby, M. T.; Halpern, J. J. Am. Chem. Soc. 1991, 113, 589; Kitamura, M.; Tokunaga, M.; Noyori, R. J. Org. Chem. 1992, 57, 4053; Takaya, H.; Ohta, T.; Inoue. S.; Tokunaga, M.; Kitamura, M.; Noyori, R. Org. Synth. 1993, 72, 74 and Zhang, X.; Uemura, T.; Matsumura, K.; Sayo, N.; Kumobayashi, H.; Takaya, H. Synlett 1994, 501. A large number of new biarylphosphine ligands have been introduced during the past decade leading to improvements in the hydrogenation of α,β-unsaturated acids. Atropisomeric bisphosphines of the P-Phos type have shown to be particularly successful as described in: Chan, A. S. C.; Chen, C.-C.; Yang, T. K.; Huang, J. H. Inog. Chim. Acta 1995, 234, 95; Chen, C.-C.; Huang, T.-T.; Ling, C.-W.; Cao, R.; Chan, A. S. C.; Wong, W. T. Inog. Chim. Acta 1998, 270, 247; Pai, C.-C.; Lin, C.-W.; Lin, C.-C.; Chen, C.-C.; Chan, A. S. C. J. Am. Chem. Soc. 2000, 122, 11513; Qiu, L.; Qi, J.; Pai, C.-C.; Chan, S.; Zhou, Z.; Choi, M. C. K.; Chan, A. S. C. Org. Lett. 2002, 4, 4599 and Pai, C.-C.; Li, Y.-M.; Zhou, Z.-Y.; Chan, A. S. C. Tetrahedron Lett. 2002, 43, 2789. The asymmetric hydrogenation of α,β-unsaturated lactones and α,β-unsaturated esters is described in chemical journals such as Ohta, T.; Miyake, T.; Seido, N.; Kumobayashi, H.; Takaya, H. J. Org. Chem. 1995, 60, 357 and Tang, W.; Wang, W.; Zhang, X. Angew. Chem., Int. Ed. Engl. 2003, 42, 943, respectively. The asymmetric hydrogenation of α,β-unsaturated lactames is described in chemical journals such as Schmid, R.; Broger, E. A.; Cereghetti, M.; Crameri, Y.; Foricher, J.; Lalonde, M.; Müller, R. K.; Scalone, M.; Schoettel, G.; Zutter, U. Pure Appl. Chem. 1996, 68, 131.

The present invention provides a process for the chemo- and diastereoselective hydrogenation of trienes of formula (III), or salts thereof, wherein R1 and R2 are as defined for a compound of formula (I), in particular those R1 and R2 substituents in above-mentioned embodiments, by employing an appropriate hydrogenation catalyst as mentioned herein.

The hydrogenation of trienes of formula (III) can in principle proceed by a number of routes as shown in Scheme 5.

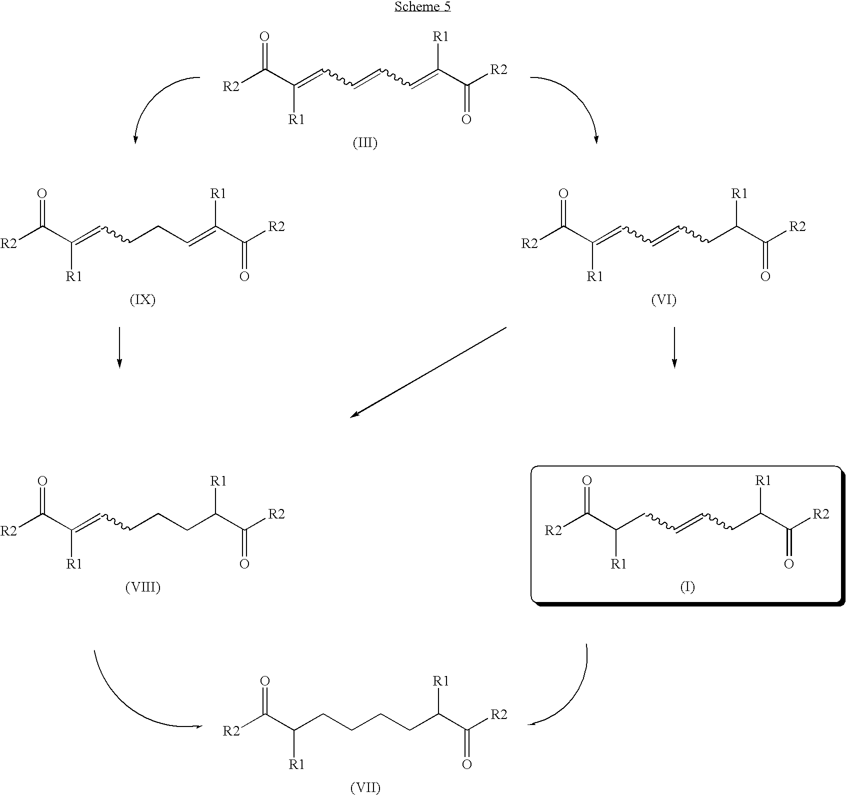

Depending upon the reactivity of each one of the double bonds and the reaction conditions, products (I) or (VI)-(IX) or mixtures thereof can be obtained. It has been found by the present inventors that the chemoselective asymmetric reduction of the “outer” double bonds of a triene of formula (III) can be achieved, for example, by the use of a ruthenium catalyst, in particular one which comprises at least a BoPhoz ligand. The BoPhoz family of ligands, which are ferrocenyl-based ligands and were developed by Boaz et al. (Boaz, N. W.; Large, S. E.; Ponasik, J. A., Jr.; Moore, M. K.; Barnette, T.; Nottingham, W. D. Org. Process Res. Dev. 2005, 9, 472), has been shown to provide important means for highly enantioselective hydrogenation reactions (Boaz, N. W.; Debenham, S. D.; Mackenzie, E. B.; Large, S. E. Org. Lett. 2002, 4, 2421).

The invention also relates, as an alternative route, to a process for preparing a compound of formula (I), or a salt thereof, wherein R1 is and R2 are as defined above, said process comprising subjecting a compound of formula (IV), or a salt thereof,

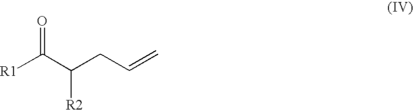

wherein R1 and R2 are as defined for a compound of formula (I), to cross-metathesis reaction to obtain a compound of formula (I), or a salt thereof.

In particular, definitions of R1 and R2 are as described before.

Starting compounds of formula (IV), or salts thereof, can be easily obtained via alkylation of a ketone 1 as shown in Scheme 1.

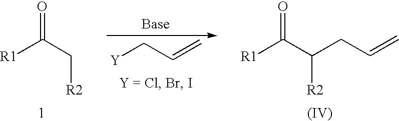

Reaction of the enolate of ketone 1, which may be prepared by the use of a base, such as lithium diisopropylamide, lithium hexamethyldisilazide, sodium hexamethyldisilazide, potassium hexamethyldisilazide or lithium 2,2,6,6-tetramethylpiperidine, with an allyl halide, such as allyl bromide, can give the compound of formula (IV).

In one particular embodiment of this metathesis approach for the preparation of compounds of formula (I), a starting compound of formula (IV), wherein R1=(S)-4-benzyl-2-oxazolidinone, can be prepared by following said reaction. The resulting compound of formula (IV) can then be submitted to cross-metathesis reaction to provide the corresponding compound of formula (I). Said compound of formula (IV) can also be converted into an ester derivative, e.g. R1=OMe or OEt, by hydrolysis, e.g. upon treatment with LiOH/H₂O₂, followed by treatment with thionyl chloride and subsequent reaction with an alcohol, e.g. MeOH or EtOH; according to methods well known to practitioners skilled in the art.

The cross-metathesis reaction of compounds of formula (IV), or salts thereof, wherein R1 and R2 are as defined above, in particular, takes place under the same conditions mentioned in embodiments for compounds of formula (II). Therefore, particular embodiments described in the prior cross-metathesis approach are also particular embodiments of this alternative cross-metathesis approach. In one embodiment, the ruthenium alkylidene catalyst is selected from entries 2a, 2b, 2d-f, 3a-c, 4a-b, 5b, 6a; in particular, 2d, 2f, 4a, 5b and 6.

Still another important aspect of the invention relates to processes for preparing compounds of formula (I), or salts thereof, wherein the metathesis step occurs in an intra-molecular fashion. Accordingly, the intra-molecular version of the first metathesis approach is also an embodiment of the present invention. Specifically, the present invention also relates to a process for preparing a compound of formula (I), or a salt thereof, wherein R1 and R2 are as described earlier, said process comprising one or more of the following steps:
a) subjecting a compound of formula (IIa), or a salt thereof,

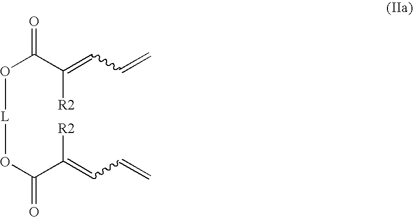

wherein
L is a linker connecting the two oxygen atoms via a 1 to 6 carbon backbone and
R2 is as defined for a compound of formula (I), to cross-metathesis reaction to obtain a compound of formula (IIIb), or a salt thereof,

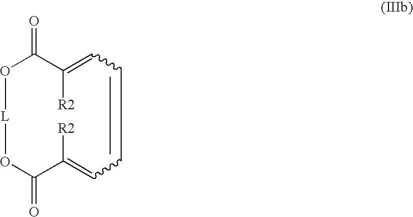

wherein L and R2 are as defined for said compound of formula (IIa);
b) converting said compound of formula (IIIb), or a salt thereof, into a compound of formula (I), or a salt thereof, by either submitting said compound of formula (IIIb), or a salt thereof, to hydrogenation followed by hydrolysis or to hydrolysis followed by hydrogenation.

In another embodiment, the second step of said process involves hydrolysis of a compound of formula (IIIb), or a salt thereof, followed by hydrogenation to obtain a compound of formula (I), or a salt thereof.

In one particular embodiment of this approach, as shown in Scheme 6, the starting compound of formula (II), wherein R1=OH and R2=isopropyl can be converted into a compound of formula (III) by following a four step protocol. First, said compound of formula (III) can be transformed into the acid chloride of formula (III), for example by treatment with oxalyl chloride. Next, said acid chloride can be reacted with 2,2′-biphenyldiol to give the corresponding compound of formula (IIa), which can then be submitted to ring closing-metathesis reaction, for example by the use of Grubbs' second generation catalyst, to obtain a compound of formula (IIIb). Finally, basic hydrolysis of such compound of formula (IIIb), according to methods well known to practitioners skilled in the art, can afford a compound of formula (III), wherein R1=OH and R2=isopropyl. Conversion of said compound into a compound of formula (I) can thus be accomplished by hydrogenation reaction, as described earlier.

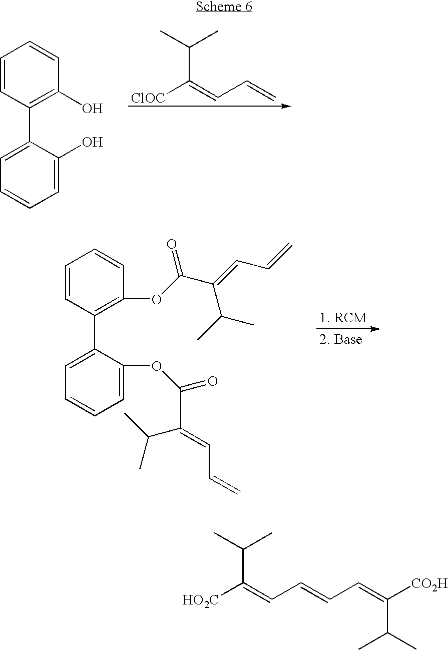

The ring-closing metathesis reaction of compounds of formula (IIa), or salts thereof, wherein R1 and R2 are as defined above for compounds of formula (I), in particular takes place under the same conditions mentioned for compounds of formula (II). Therefore, particular embodiments described in the first cross-metathesis approach are also particular embodiments of this first ring-closing metathesis approach. Inone embodiment, the ruthenium alkylidene catalysts is Grubbs' second generation catalyst.

Similarly, the intra-molecular version of the second metathesis approach is also an embodiment of the present invention. Specifically, the present invention also relates to a process for preparing a compound of formula (I), or a salt thereof, wherein R1 and R2 are as described earlier, said process comprising one or more of the following steps:
a) subjecting a compound of formula (IVa), or a salt thereof,

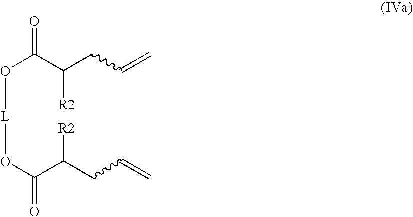

wherein
L is a linker connecting the two oxygen atoms via a 1 to 6 carbon backbone and
R2 is as defined for a compound of formula (I), to cross-metathesis reaction to obtain a compound of formula (Ic), or a salt thereof,

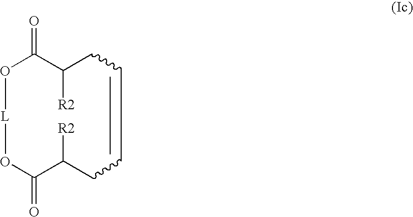

wherein L and R2 are as defined for said compound of formula (IVa);
b) converting said compound of formula (Ic), or a salt thereof, into a compound of formula (I), or a salt thereof, by hydrolysis reaction.

The ring-closing metathesis reaction of compounds of formula (IVa), or salts thereof, wherein R1 and R2 are as defined above for compounds of formula (I), in particular takes place under the same conditions mentioned for compounds of formula (IV). Therefore, particular embodiments described in the second cross-metathesis approach are also particular embodiments of this second ring-closing metathesis approach. In one embodiment, the ruthenium alkylidene catalysts is 2a.

The linker for compounds of formulae (IIa), (IIIb), (IVa) and (Ic) is as defined herein and is for example selected from the group consisting of:
a) an unsubstituted or substituted, C₁₋₆alkylene chain, in particular; C₄₋₆alkylene chain
b) an unsubstituted or substituted, C₄₋₈cycloalkylene, in particular; C₆₋₈cycloalkylene
c) an unsubstituted or substituted heterocyclylene, in particular; N-(unsubstituted or substituted)aryl pyrrolidinylene or N-(unsubstituted or substituted)aryl pyrrolidinedionylene.
d) the biradical of formula (X)

—(CH₂)ₖ-A-(CH₂)ₗ—Bₘ—(CH₂)ₙ— (X)
wherein
k, l and n are independently 0, 1 or 2;
m is 0 or 1;
A and B are independently, unsubstituted or substituted, aryl or heteroaryl, for example phenyl; connected, independently, in an ortho, para or meta fashion, in particular meta or ortho. In one embodiment, biradicals of formula (X) are -A-(CH₂)ₗ—Bₘ— or —(CH₂)ₖ-A-(CH₂)ₗ, in particular —CH₂-A-CH₂—, or -A- or -A-B—.

In particular linkers for compounds of formulae (IIa), (IIIb), (IVa) and (Ic) are selected from the following moieties, wherein the asterisk (*) denotes the point of binding to one of the oxygen atoms,

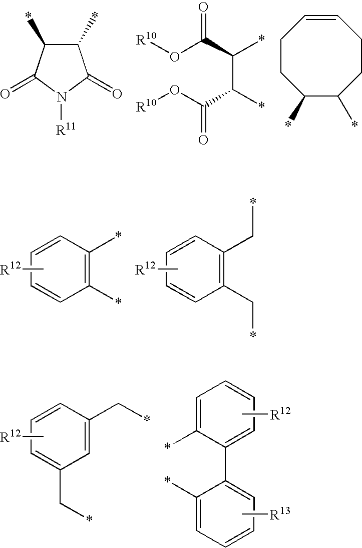

and wherein;
R10 is hydrogen, C₁₋₇alkyl, phenyl- or naphthyl-C₁₋₄alkyl, aryl or C₃₋₈cycloalkyl, each unsubstituted or substituted by halo, dialkylamino, nitro, halo-C₁-C₇-alkyl, C₁-C₇-alkyl, C₁-C₇alkoxy, halo-C₁-C₇-alkoxy, such as trifluoromethoxy, or C₁-C₇-alkoxy-C₁-C₇-alkoxy; R11 is C₁₋₇alkyl, phenyl- or naphthyl-C₁₋₄alkyl, aryl or C₃₋₈cycloalkyl, each unsubstituted or substituted by halo, dialkylamino, nitro, halo-C₁-C₇-alkyl, C₁-C₇-alkoxy, halo-C₁-C₇-alkoxy, such as trifluoromethoxy, and C₁-C₇-alkoxy-C₁-C₇-alkoxy; and,
R12 and R13 are independently selected from the group of hydrogen, halo, dialkylamino, nitro, halo-C₁-C₇-alkyl, C₁-C₇alkoxy, halo-C₁-C₇-alkoxy, such as trifluoro-methoxy, and C₁-C₇-alkoxy-C₁-C₇-alkoxy.

Each of the above mentioned olefin metathesis strategies can be used individually in a method to prepare renin inhibitors such as aliskiren.

Compounds of formula (I) or salts thereof, can be converted into aliskiren, or a salt thereof. As shown in Scheme 7, a starting compound of formula (I) can be converted into a compound of formula (XIV).

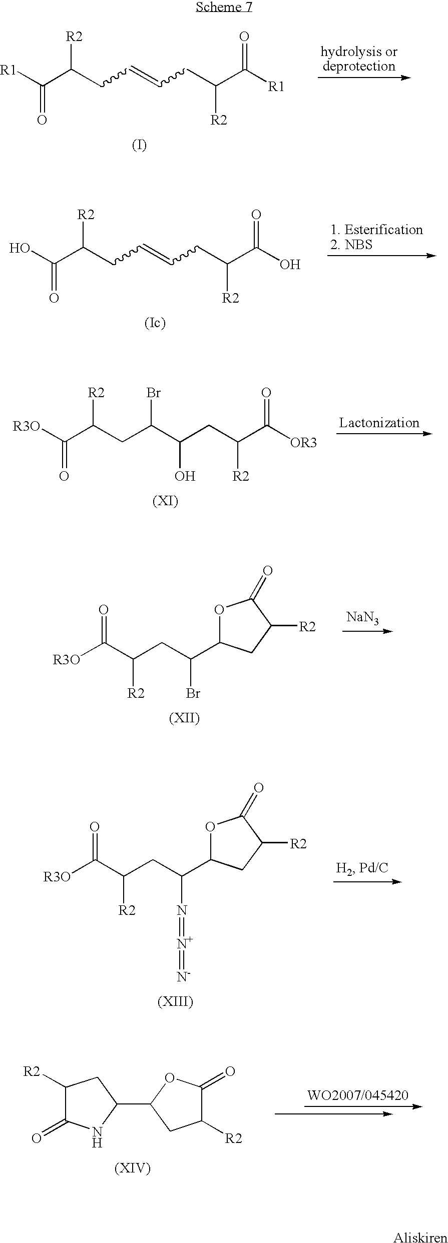

According to Scheme 7, said compound of formula (I), or salt thereof, wherein R1 and R2 are as defined earlier, can be converted into a compound of formula (Ic) via hydrolysis or deprotection methods well known to practitioners skilled in the art. Standard conditions for such methods are described, for example, in relevant chapters in J. F. W. McOmie, “Protective Groups in Organic Chemistry”, Plenum Press, London and New York 1973, in T. W. Greene and P. G. M. Wuts, “Protective Groups in Organic Synthesis”, Third edition, Wiley, New York 1999 and in Richard C. Larock, “Comprehensive Organic Transformations: A Guide to Functional Group Preparations”, Second Edition, Wiley-VCH Verlag GmbH, 2000. A compound of formula (Ic), or salt thereof, can then be transformed into a compound of formula (XI), or salt thereof, wherein R3 is as defined earlier, for example by treatment with MeI and K₂CO₃ (R3=Me) followed by treatment with N-bromosuccinimide. Next, lactonization and subsequent bromide displacement with an azide, for example by using sodium azide, can afford an azido lactone of formula (XIII), or salt thereof. Hydrogenation of an azido lactone of formula (XIII), or salt thereof, for example with hydrogen in the presence of palladium on charcoal, can afford a lactone-lactam of formula (XIV), or salt thereof. Finally, the lactone-lactam of formula (XIV), or a salt thereof, wherein R2 is as defined for a compound of formula (I), in particular R2 is isopropyl, may be used for the synthesis of renin inhibitors, in particular renin inhibitors comprising a 2,7-dialkyl-4-hydroxy-5-amino-8-aryl-octanoyl amide backbone, such as aliskiren, or a salt thereof, as described e.g. in WO2007/045420., in particular in the claims and Examples.

Alternatively, a compound of formula (I), or salt thereof, can be converted into the key lactone-lactam of formula (XIV), or salt thereof, as described in Scheme 8.

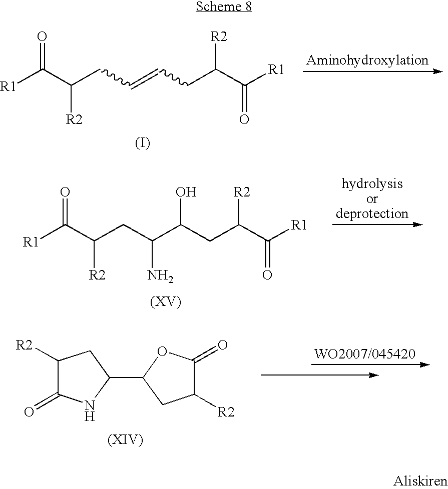

Specifically, a compound of formula (I), or a salt thereof, wherein R1 and R2 are as defined earlier, can be first subjected to aminohydroxylation, for example under Sharpless' conditions (M. A. Andersson, R. Epple, V. V. Fokin and K. B. Sharpless, Angew. Chem. Int. Ed., 41, 472, 2002). Upon aminohydroxylation, the resulting amino alcohol of formula (XV), or salt thereof, wherein R1 and R2 are as defined above, can be transformed onto the lactone-lactam of formula (XIV), or salt thereof, via hydrolysis or deprotection. The deprotection step of compounds of formula (XV), or salts thereof, wherein R1 and R2 are as previously defined, can proceed under standard conditions and as described in relevant chapters of reference books such as J. F. W. McOmie, “Protective Groups in Organic Chemistry”, Plenum Press, London and New York 1973, in T. W. Greene and P. G. M. Wuts, “Protective Groups in Organic Synthesis”, Third edition, Wiley, New York 1999. The hydrolysis step of compounds of formula (XV), or salts thereof, wherein R1 and R2 are as previously defined, can proceed under standard conditions and as described in relevant chapters of reference books such as Richard C. Larock, “Comprehensive Organic Transformations: A Guide to Functional Group Preparations”, Second Edition, Wiley-VCH Verlag GmbH, 2000.

In one embodiment, compounds of formulae (XI)-(XV), or salts thereof, have the following stereochemistry:

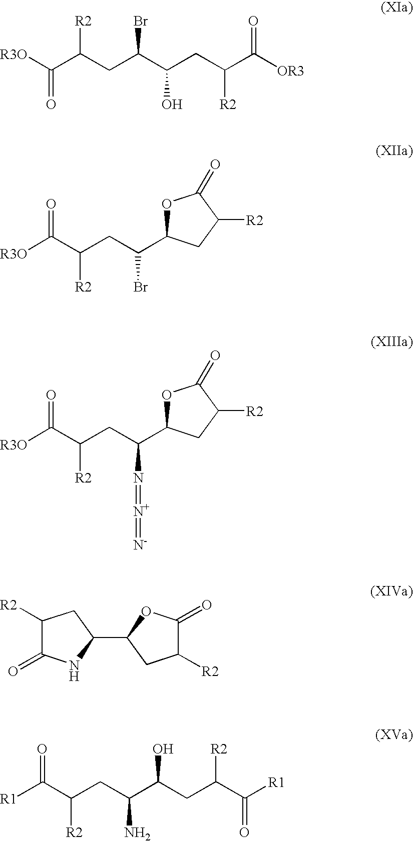

R1, R2 and R3 groups for compounds of formulae (XIa)-(XVa) are as defined above. In particular R3 is methyl. In particular, R2 is isopropyl.

In another embodiment, compounds of formulae (XI)-(XV), or salts thereof, have the following stereochemistry:

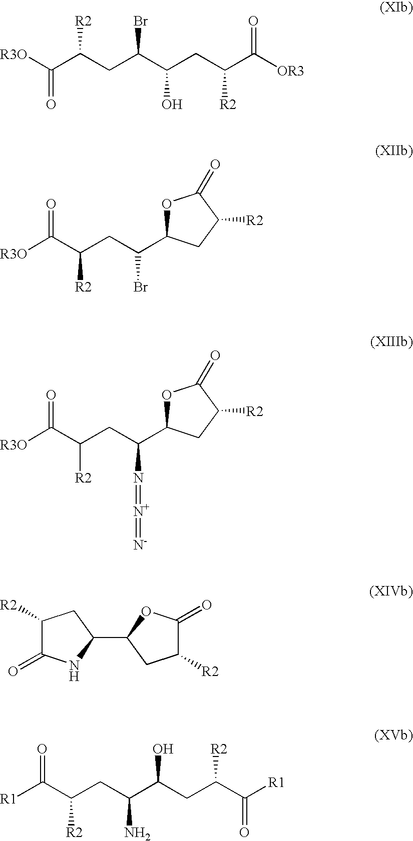

R1, R2 and R3 groups for compounds of formulae (XIb)-(XVb) are as defined above. In particular, R3 is methyl. In particular, R2 is isopropyl.

In a particular embodiment, the starting compound of formula (I), in Schemes 7 or 8, is (S,S)-(E)-2,7-diisopropyl-4-octene-1,8-dioic acid, or the salt thereof [i.e. a compound of formula (Ib) wherein R1=OH and R2=isopropyl, or the salt thereof].

In another embodiment, the present invention relates to a process for preparing a compound of formula (XVI)

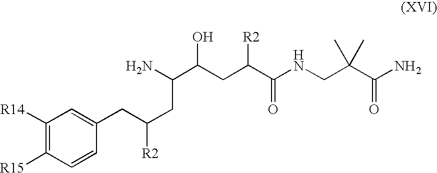

wherein R2 is as defined for a compound of formula (I), R14 is halogen, hydroxyl, C₁₋₆halogenalkyl, C₁₋₆alkoxy-C₁₋₆alkyloxy or C₁₋₆alkoxy-C₁₋₆alkyl; R15 is halogen, hydroxyl, C₁₋₄alkyl or C₁₋₄alkoxy, or a salt thereof, comprising one or more of the following steps either individually or in any combination:
the manufacture of a compound of the formula III, as defined herein, by treating, as defined above, a compound of the formula II, as defined herein;
the manufacture of a compound of the formula I, as defined herein, by treating, as defined above, a compound of the formula III, as defined herein;
the manufacture of the above compound of the formula XVI, in particular wherein the compound the formula XVI is aliskiren, by treating, as defined above, a compound of the formula I, as defined herein.

In yet another embodiment, the present invention relates to a process for preparing the compound of formula (XVI) as defined above, comprising one or more of the following steps either individually or in any combination:
the manufacture of a compound of the formula IIIb, as defined herein, by treating, as defined above, a compound of the formula IIa, as defined herein;
the manufacture of a compound of the formula I, as defined herein, by treating, as defined above, a compound of the formula IIIb, as defined herein;
the manufacture of the above compound of the formula XVI, in particular wherein the compound the formula XVI is aliskiren, by treating, as defined above, a compound of the formula I, as defined herein.

In still another embodiment, the present invention relates to a process for preparing the compound of formula (XVI) as defined above, comprising one or more of the following steps either individually or in any combination:
the manufacture of a compound of the formula I, as defined herein, by treating, as defined above, a compound of the formula IV, as defined herein;
the manufacture of the above compound of the formula XVI, in particular wherein the compound the formula XVI is aliskiren, by treating, as defined above, a compound of the formula I, as defined herein.

In a further embodiment, the present invention relates to a process for preparing the compound of formula (XVI), as defined above, comprising one or more of the following steps either individually or in any combination:
the manufacture of a compound of the formula Ic, as defined herein, by treating, as defined above, a compound of the formula IVa, as defined herein;
the manufacture of a compound of the formula I, as defined herein, by treating, as defined above, a compound of formula Ic, as defined herein;
the manufacture of the above compound of the formula XVI, in particular wherein the compound the formula XVI is aliskiren, by treating, as defined above, a compound of formula I, as defined herein.

According to an aspect of the present invention, there are provided chemical compounds of the formulae (XI), (XII), (XIII) and (XV), or salts thereof, useful as intermediates in the preparation of other compounds which may, in turn, be used as valuable starting materials for the production of pharmaceutically active compounds. Specifically, compounds of the formulae (XI), (XII), (XIII) and (XV), or salts thereof, are useful as intermediates in the preparation of compounds of formula (XIV), or a salts thereof, which are intermediates in the preparation of renin inhibitors, in particular renin inhibitors comprising a 2,7-dialkyl-4-hydroxy-5-amino-8-aryl-octanoyl amide backbone, such as aliskiren or a pharmaceutically acceptable salt thereof. Compounds of formulae (XIa), (XIIa), (XIIIa) and (XVa), or salts thereof are embodiments of the invention. Compounds of formulae (XIb), (XIIb), (XIIIb) and (XVb), or salts thereof are further embodiments of the invention.

Still another important aspect of the invention relates to new processes for preparing compounds of formula (XIV), or salts thereof. In one embodiment, the invention relates to processes for preparing compounds of formula (XIVa), or salts thereof, in another embodiment processes for preparing compounds of formula (XIVb), or salts thereof.

According to a still further aspect of the present invention, there are provided chemical compounds of the formulae (IIa), (IIIb), (IVa) and (Ic), or salts thereof, useful as intermediates in the preparation of other compounds which may, in turn, be used as valuable starting materials for the production of pharmaceutically active compounds. Specifically, compounds of the formulae (IIa), (IIIb), (IVa) and (Ic), or salts thereof, are useful as intermediates in the preparation of compounds of formula (I), or a salt thereof, which are intermediates in the preparation of renin inhibitors, in particular renin inhibitors comprising a 2,7-dialkyl-4-hydroxy-5-amino-8-aryl-octanoyl amide backbone, such as aliskiren or a pharmaceutically acceptable salt thereof.

Listed below are definitions of various terms used to describe the novel intermediates and synthesis steps of the present invention. These definitions, either by replacing one, more than one or all general expressions or symbols used in the present disclosure and thus yielding embodiments of the invention, in particular apply to the terms as they are used throughout the specification unless they are otherwise limited in specific instances either individually or as part of a larger group.

The term “C₁-C₇” defines a moiety with up to and including maximally 7, in particular up to and including maximally 4, carbon atoms, said moiety being branched (one or more times) or straight-chained and bound via a terminal or a non-terminal carbon

The term alkyl, as a radical or part of a radical, defines a moiety with up to and including maximally 7, C₁₋₇alkyl, in particular up to and including maximally 4, C₁₋₄alkyl, carbon atoms, said moiety being branched (one or more times) or straight-chained and bound via a terminal or a non-terminal carbon. Lower or C₁-C₇alkyl, for example, is n-pentyl, n-hexyl or n-heptyl or in particular C₁-C₄-alkyl, for example methyl, ethyl, n-propyl, sec-propyl, i-propyl, n-butyl, isobutyl, sec-butyl and tert-butyl. Very preferred is iso-propyl.

Branched alkyl in particular comprises 3 to 6 C atoms. Examples are i-propyl, i- and t-butyl, and branched isomers of pentyl and hexyl.

halo-C₁-C₇-alkyl may be linear or branched and in particular comprises 1 to 4 C atoms, for example 1 or 2 C atoms. Examples are fluoromethyl, difluoromethyl, trifluoromethyl, chloromethyl, dichloromethyl, trichloromethyl, 2-chloroethyl and 2,2,2-trifluoroethyl.

The term “C₃₋₈cycloalkyl”, as a radical or part of a radical, defines a cycloalkyl moiety with up to and including maximally 8, in particular up to and including maximally 6, carbon atoms. Said cycloalkyl moiety is for example mono- or bicyclic, in particular monocyclic, which may include one or more double and/or triple bonds and, is unsubstituted or substituted by one or more, e.g. one to four substitutents. Embodiments include a cyclopropyl, cyclobutyl, cyclopentyl, cyclohexyl, cycloheptyl or cyclooctyl, which is unsubstituted or substituted. Substituents are, for example, selected from the group of hydroxyl, halo, oxo, amino, alkylamino, dialkylamino, thiol, alkylthio, nitro, hydroxy-C₁-C₇-alkyl, C₁-C₇-alkanoyl, such as acetyl, C₁-C₇-alkoxy, halo-C₁-C₇-alkoxy, such as trifluoromethoxy, hydroxy-C₁-C₇-alkoxy, and C₁-C₇-alkoxy-C₁-C₇alkoxy, carbamoyl and cyano.

Unsubstituted or substituted aryl, as a radical or part of a radical, for example is a mono- or bicyclic aryl with 6 to 22 carbon atoms, such as phenyl, indenyl, indanyl or naphthyl, in particular phenyl, and is unsubstituted or substituted by one or more, for example one to three, substitutents, in particular, independently selected from the substitutents mentioned above for cycloalkyl.

Substituted phenyl- or naphthyl-C₁-C₄-alkyl refers to a C₁-C₄-alkyl wherein the phenyl- or naphthyl- is substituted by one or more, for example one to three, substitutents, for example, independently selected from the substitutents mentioned above for cycloalkyl.

The 3 to 7 membered nitrogen containing saturated hydrocarbon ring formed by R4 and R5, which can be unsubstituted or substituted, is for example unsubstituted or substituted by one or more, e.g. one to four substitutents in particular independently selected from those mentioned above as substituents for cycloalkyl, for example a 4- to 7-membered ring that is unsubstituted or substituted by up to four substituents, such as one substituent, selected for example from hydroxy, halo, such as chloro, C₁-C₇-alkyl, such as methyl, cyano, hydroxy-C₁-C₇-alkyl, halo-C₁-C₇-alkyl, C₁-C₇-alkanoyl, such as acetyl, C₁-C₇-alkoxy, halo-C₁-C₇-alkoxy, such as trifluoromethoxy, hydroxy-C₁-C₇-alkoxy, and C₁-C₇-alkoxy-C₁-C₇-alkoxy; in particular an oxazolidinone or piperidine ring is formed by R4 and R5 that is unsubstituted or substituted by up to four moieties selected from C₁-C₇-alkyl, aryl-C₁-C₇-alkyl, hydroxyl, halo, hydroxy-C₁-C₇-alkyl, halo-C₁-C₇-alkyl and cyano, in one embodiment an oxazolidinone is formed by R4 and R5 that is unsubstituted or substituted by up to four moieties selected from C₁-C₇-alkyl, substituted aryl-C₁-C₇-alkyl, hydroxyl, halo, hydroxy-C₁-C₇-alkyl, halo-C₁-C₇-alkyl and cyano, or a piperidine is formed by R4 and R5 that is unsubstituted or substituted by up to four moieties selected from C₁-C₇-alkyl, aryl-C₁-C₇-alkyl, hydroxyl, halo, hydroxy-C₁-C₇-alkyl, halo-C₁-C₇-alkyl and cyano,

Silyl is —SiRR′R″, wherein R, R′ and R″ are independently of each other C₁₋₇alkyl, aryl or phenyl-C₁₋₄alkyl.

Alkanoyl is, for example, C₁-C₇-alkanoyl and is, for example, acetyl [—C(═O)Me], propionyl, butyryl, isobutyryl or pivaloyl, in particular C₂-C₅-Alkanoyl, for example acetyl.

Alkoxy being a radical or part of a radical is, for example, C₁-C₇-alkoxy and is, for example, methoxy, ethoxy, n-propyloxy, isopropyloxy, n-butyloxy, isobutyloxy, sec-butyloxy, tert-butyloxy and also includes corresponding pentyloxy, hexyloxy and heptyloxy radicals, in particular C₁-C₄alkoxy. Alkoxy may be linear or branched and in particular comprises 1 to 4 C atoms. Examples are methoxy, ethoxy, n- and i-propyloxy, n-, i- and t-butyloxy, pentyloxy and hexyloxy.

halo-C₁-C₇alkoxy may be linear or branched. Examples are trifluoromethoxy and trichloromethoxy.

Alkoxyalkyl may be linear or branched. The alkoxy group for example comprises 1 to 7 and in particular 1 or 4 C atoms, and the alkyl group for example comprises 1 to 7 and in particular 1 or 4 C atoms. Examples are methoxymethyl, 2-methoxyethyl, 3-methoxypropyl, 4-methoxybutyl, 5-methoxypentyl, 6-methoxyhexyl, ethoxymethyl, 2-ethoxyethyl, 3-ethoxypropyl, 4-ethoxybutyl, 5-ethoxypentyl, 6-ethoxyhexyl, propyloxymethyl, butyloxymethyl, 2-propyloxyethyl and 2-butyloxyethyl.

Alkylamino and dialkylamino may be linear or branched. The alkyl group for example comprises 1 to 7 and in particular 1 or 4 C atoms. Some examples are methylamino, dimethylamino, ethylamino, and diethylamino.

Alkylthio may be linear or branched. The alkyl group for example comprises 1 to 7 and in particular 1 or 4 C atoms. Some examples are methylthio and ethylthio.

C₁₋₆alkylene is a bivalent radical derived from C₁₋₆alkyl and is especially C₂-C₆-alkylene or C₂-C₆-alkylene which is interrupted by, one or more, e.g one or two, C═C, which may be part of an aryl or heterorayl moiety, O, NRx or S, wherein Rx is C₁₋₇alkyl, unsubstituted or substituted phenyl- or naphthyl-C₁₋₄alkyl, unsubstituted or substituted aryl or unsubstituted or substituted C₃₋₈cycloalkyl, wherein substituted refers to one or more, for example one to three, substitutents in particular independently selected from the substitutents mentioned above for cycloalkyl. The C₁₋₆alkylene may be unsubstituted or substituted by one or more, for example one to three, substitutents, in particular, independently selected from the substitutents mentioned above for cycloalkyl.

C₄₋₈cycloalkylene is a bivalent radical derived from C₄₋₈alkyl and is especially C₂-C₆-alkylene or C₂-C₆-alkylene which is interrupted by, one or more, e.g one or two, C═C, which may be part of an aryl or heterorayl moiety, O, NRx or S, wherein Rx is C₁₋₇alkyl, unsubstituted or substituted phenyl- or naphthyl-C₁₋₄alkyl, unsubstituted or substituted aryl or unsubstituted or substituted C₃₋₈cycloalkyl, wherein substituted refers to one or more, for example one to three, substitutents in particular independently selected from the substitutents mentioned above for cycloalkyl. The C₄₋₈cycloalkylene may be unsubstituted or substituted by one or more, for example one to three, substitutents, in particular independently selected from the substitutents mentioned above for cycloalkyl.

Heterocyclylene is a bivalent radical derived from heterocyclyl, as defined herein, and is in particular N-(unsubstituted or substituted)aryl pyrrolidinylene or N-(unsubstituted or substituted)aryl pyrrolidinedionylene.

In formulae above the term [symbol] represents a covalent bond, which comprises an (E) stereoisomer as well as a (Z) stereoisomer of the respective olefin.

Terms d,l and meso are used herein following stereodescriptor nomenclature according to: Gutsche, C. D.; Pasto, D. J. Fundamentals of Organic Chemistry, Prentice-Hall, Inc., Englewood Cliffs, N.J., 1975 and, Eliel, E. L.; Wilen, S. H. Stereochemistry of Organic Compounds, John Wiley & Sons, Inc. 1994.

Halo or halogen is for example fluoro, chloro, bromo or iodo, in particular fluoro, chloro or bromo; where halo is mentioned, this can mean that one or more (e.g. up to three) halogen atoms are present, e.g. in halo-C₁-C₇alkyl, such as trifluoromethyl, 2,2-difluoroethyl or 2,2,2-trifluoroethyl.

Unsubstituted or substituted heterocyclyl is a mono- or polycyclic, for example a mono-, bi- or tricyclic-, such as mono-, unsaturated, partially saturated, saturated or aromatic ring system with for example 3 to 22 (in particular 3 to 14) ring atoms and with one or more, for example one to four, heteroatoms independently selected from nitrogen, oxygen, sulfur, S(═O)— or S-(═O)₂, and is unsubstituted or substituted by one or more, e.g. up to three, substitutents, for example, independently selected from the substitutents mentioned above for cycloalkyl. When the heterocyclyl is an aromatic ring system, it is also referred to as heteroaryl.

Alkylene chain, C₄₋₈cycloalkylene, heterocyclylene are bivalent radicals derived from C₁₋₇alkyl, C₄₋₈cycloalkyl and heterocyclyl, respectively, and are unsubstituted or substituted by one or more, e.g. up to three, substitutents, for example, independently selected from the substitutents mentioned above for cycloalkyl.

Salts are in particular pharmaceutically acceptable salts or generally salts of any of the intermediates mentioned herein, where salts are not excluded for chemical reasons the skilled person will readily understand. They can be formed where salt forming groups, such as basic or acidic groups, are present that can exist in dissociated form at least partially, e.g. in a pH range from 4 to 10 in aqueous solutions, or can be isolated for example in solid, in particular crystalline, form.

Such salts are formed, for example, as acid addition salts, for example with organic or inorganic acids, from compounds or any of the intermediates mentioned herein with a basic nitrogen atom (e.g. imino or amino), in particular the pharmaceutically acceptable salts. Suitable inorganic acids are, for example, halogen acids, such as hydrochloric acid, sulfuric acid, or phosphoric acid. Suitable organic acids are, for example, carboxylic, phosphonic, sulfonic or sulfamic acids, for example acetic acid, propionic acid, lactic acid, fumaric acid, succinic acid, citric acid, amino acids, such as glutamic acid or aspartic acid, maleic acid, hydroxymaleic acid, methylmaleic acid, benzoic acid, methane- or ethane-sulfonic acid, ethane-1,2-disulfonic acid, benzenesulfonic acid, 2-naphthalenesulfonic acid, 1,5-naphthalene-disulfonic acid, N-cyclohexylsulfamic acid, N-methyl-, N-ethyl- or N-propyl-sulfamic acid, or other organic protonic acids, such as ascorbic acid.

In the presence of negatively charged radicals, such as carboxy or sulfo, salts may also be formed with bases, e.g. metal or ammonium salts, such as alkali metal or alkaline earth metal salts, for example sodium, potassium, magnesium or calcium salts, or ammonium salts with ammonia or suitable organic amines, such as tertiary monoamines, for example triethylamine or tri(2-hydroxyethyl)amine, or heterocyclic bases, for example N-ethyl-piperidine or N,N′-dimethylpiperazine.

When a basic group and an acid group are present in the same molecule, any of the intermediates mentioned herein may also form internal salts.

For isolation or purification purposes of any of the intermediates mentioned herein it is also possible to use pharmaceutically unacceptable salts, for example picrates or perchlorates.

In view of the close relationship between the compounds and intermediates in free form and in the form of their salts, including those salts that can be used as intermediates, for example in the purification or identification of the compounds or salts thereof, any reference to “compounds”, “starting materials” and “intermediates” hereinbefore and hereinafter is to be understood as referring also to one or more salts thereof or a mixture of a corresponding free compound, intermediate or starting material and one or more salts thereof, each of which is intended to include also any solvate or salt of any one or more of these, as appropriate and expedient and if not explicitly mentioned otherwise. Different crystal forms may be obtainable and then are also included.

Where the plural form is used for compounds, starting materials, intermediates, salts, pharmaceutical preparations, diseases, disorders and the like, this is intended to mean one (in particular) or more single compound(s), salt(s), pharmaceutical preparation(s), disease(s), disorder(s) or the like, where the singular or the indefinite article (“a”, “an”) is used, this is not intended to exclude the plural, but only preferably means “one”.

The following Examples serve to illustrate the invention without limiting the scope thereof, while they on the other hand represent particular embodiments of the reaction steps, intermediates and/or the process of manufacture of aliskiren, or salts thereof.

### ABBREVIATIONS

δ chemical shift
μl microlitre
Ac acetyl
Bn benzyl
Boc tert-butoxycarbonyl
br broad
brm broad multiplet
n-BuLi butyl lithium
DCM dichloromethane
de diastereomeric excess
DMAP 4-(dimethylamino)pyridine

### DMF N,N-dimethylformamide

DMSO dimethylsulfoxide
ee enantiomeric excess
equiv equivalent
ES electrospray
ESI electrospray ionisation
Et ethyl
EtOAc ethyl acetate
FTIR fourier transform infrared spectroscopy
GC gass chromatography
h hour(s)
HCl hydrogen chloride
HNMR proton nuclear magnetic resonance
H₂O₂ hydrogen peroxide
HPLC high performance liquid chromatography
i-Pr isopropyl
iPrOAc isopropyl acetate
IR infrared
K₂CO₃ potassium carbonate
KHMDS potassium bis(trimethylsilyl)amide
L litre
LCMS liquid chromatography-mass spectrometry
LDA lithium diisopropylamide
LHMDS lithium bis(trimethylsilyl)amide
LiOH lithium hydroxide
LRMS low resolution mass spectroscopy
M molarity
m/e mass-to-charge ratio
Me methyl
MeOH methanol
mg milligram
MgSO₄ magnesium sulfate
min minute(s)
mL millilitre
mmol(s) millimole(s)
mol(s) mole(s)
mp melting point
MS mass spectrometry
MTBE tertbutylmethylether
NaCl sodium chloride
NaH sodium hydride
NaHCO₃ sodium bicarbonate
NH₄Cl ammonium chloride
NaHMDS sodium bis(trimethylsilyl)amide
NaOMe sodium methoxide
Na₂SO₃ sodium sulfite
nm nanometre
NMR nuclear magnetic resonance
Pd/C palladium on carbon
Ph phenyl
Piv pivaloyl
ppm parts per million
psi pounds per square inch
RT room temperature
SiO₂ silica
TBDMS tertbutyldimethylsilyl
TES triethylsilyl
TFA trifluoroacetic acid
THF tetrahydrofuran
TLC thin layer chromatography

### TMEDA N,N,N,N-tetramethylethylenediamine

TMS trimethylsilyl
t_{R} retention time
Ts tosylate/tosyl

### EXAMPLES

### Ethyl 3-hydroxy-2-isopropyl-4-pentenoate (2A)

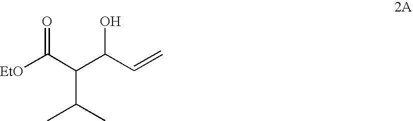

To a stirred solution of diisopropylamine (33.6 mL, 240 mmol) in dry THF (140 mL) at −78° C. is added n-BuLi (138 mL, 1.6 M in hexanes, 220 mmol) and the solution is stirred at 0° C. for 30 minutes. Ethyl isovalerate (30 mL, 200 mmol) is then added at −78° C. and the solution is stirred for 30 additional minutes at this temperature. Acrolein (14.7 mL, 220 mmol) is added at −78° C. and the mixture is further stirred for 1 hour. The solution is then quenched by addition of saturated aqueous NH₄Cl (500 mL) and allowed to warm to room temperature. The aqueous phase is extracted with EtOAc (500 mL) and the combined organic phases are washed with water and brine, dried (MgSO₄) and evaporated to dryness to give 2A as a brown oil that is used directly into the next step. ¹H NMR (400.13 MHz, CDCl₃) δ 0.90 (d, 3H, J=6.8 Hz), 0.92 (d, 3H, J=6.8 Hz), 1.20 (t, 3H, J=7.1 Hz), 2.0-2.1 (m, 1H), 2.35 (t, 1H, J=6.8 Hz), 4.0-4.1 (m, 2H), 4.33 (t, J=6.7 Hz, 1H), 5.11 (d, 1H, J=10.4 Hz), 5.23 (dt, 1H, J=17.2, 1.2 Hz), 5.89 (ddd, 1H, J=17.1, 10.4, 6.7 Hz) ppm.

### Ethyl 2-isopropyl-3-methanesulfonyloxy-4-pentenoate (3A)

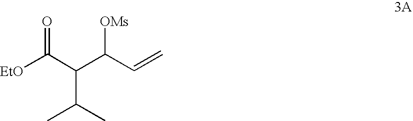

To a stirred solution of 2A (37.2 g, 200 mmol) in dry THF (500 mL) at 0° C. are subsequently added Et₃N (59.6 mL, 420 mmol) and methanesulfonyl chloride (17.2 mL, 220 mmol). The mixture is stirred at room temperature for 1 hour and then diluted with EtOAc (500 mL), washed with water and brine, dried (MgSO₄) and evaporated to dryness to give 3A as a brown oil that is used into the next step without further purification. ¹H NMR (400.13 MHz, CDCl₃) δ 0.91 (d, 3H, J=6.8 Hz), 0.94 (d, 3H, J=6.8 Hz), 1.19 (t, 3H, J=7.1 Hz), 1.9-2.1 (m, 1H,), 2.59 (dd, 1H, J=8.2), 2.92 (s, 1H,), 4.08 (q, 2H, J=7.1 Hz), 5.18 (t, J=8.3 Hz, 1H), 5.35 (d, 1H, J=10.3 Hz), 5.43 (d, 1H, J=17.2 Hz), 5.98 (ddd, 1H, J=17.2, 10.3, 8.5 Hz) ppm.

### Ethyl (E)-2-isopropyl-2,4-pentadienoate (IIA)

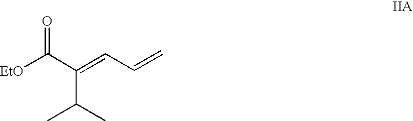

NaOMe (400 mL, 1M in MeOH, 400 mmol) is added to a solution of 3A (52.8 g, 200 mmol) in dry THF (1 L) and the mixture is stirred overnight at room temperature. The solution is then diluted with EtOAc (1 L), washed with water and brine, dried (MgSO₄) and evaporated to give a brown oil. Distillation at 55-58° C. at 250 mTorr gives IIA as a light yellow oil. ¹H NMR (400.13 MHz, CDCl₃) δ 1.14 (d, 6H, J=7.0 Hz), 1.25 (t, 3H, J=7.1 Hz), 3.00 (septuplet, 1H, J=7.0 Hz), 4.13 (q, 2H, J=7.1 Hz), 5.35 (d, 1H, J=10.0 Hz), 5.47 (d, 1H, J=16.6 Hz), 6.68 (ddd, 1H, J=16.6, 11.4, 10.0 Hz), 6.94 (d, 1H, J=11.4 Hz) ppm

### (E)-2-Isopropyl-2,4-pentadienoic acid (IIB)

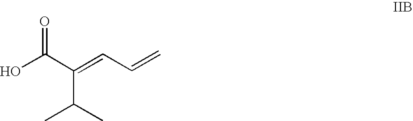

A solution of ethyl (E)-2-isopropyl-2,4-pentadienoate IIA (2.02 g, 12 mmol) in a 1:1 mixture of THF:MeOH (12 mL) is treated with a 2 M aqueous solution of LiOH (12 mL, 24 mmol) and stirred over night at 80° C. After cooling down to room temperature, the reaction mixture is diluted with water (12 mL) and washed with MTBE. The aqueous phase is then acidified by addition of 1M KHSO₄ and extracted with MTBE (3×). The combined organic phases are dried (MgSO₄) and evaporated to give (E)-2-isopropyl-2,4-pentadienoic acid IIB as an oil. ¹H NMR (400.13 MHz, CDCl₃) δ1.16 (d, 6H, J=7.0 Hz,), 3.01 (septuplet, 1H, J=7.0 Hz), 5.42 (d, 1H, J=10.3 Hz), 5.53 (d, 1H, J=16.7 Hz), 6.71 (ddd, 1H, J=16.7, 11.5, 10.3 Hz), 7.11 (d, 1H, J=11.5 Hz) ppm.

### (E)-2-Isopropyl-2,4-pentadienoic acid diisopropylamide (IIC)

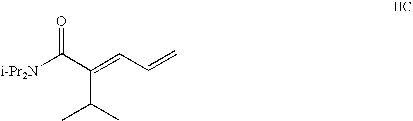

A solution of (E)-2-isopropyl-2,4-pentadienoic acid IIB (1.0 g, 7.18 mmol) in CH₂Cl₂ (15 mL) is treated with a drop of DMF followed by oxalyl chloride (0.93 mL, 10.8 mmol). After stirring for 1 hour at room temperature, the mixture is cooled to 0° C. and triethylamine (1.5 mL, 10.8 mmol) followed by diisopropylamine (1.5 mL, 10.8 mmol) are slowly added. The mixture is then warmed up to room temperature, stirred for an extra hour, and quenched by addition of saturated aqueous NaHCO₃ (10 mL). The aqueous phase is extracted with MTBE (3×), washed with 10% citric acid aqueous solution and water, dried (MgSO₄) and evaporated to give (E)-2-isopropyl-2,4-pentadienoic acid diisopropylamide IIC as a single geometric isomer. ¹H NMR (400.13 MHz, CDCl₃) [symbol] 1.01 (brs, 6H), 1.08 (d, 6H, J=7.0 Hz), 1.38 (brs, 6H), 2.92 (septuplet, 1H, J=7.0 Hz), 3.34 (brs, 1H), 4.05 (brs, 1H), 5.1-5.2 (m, 2H), 5.75 (d, 1H, J=9.0 Hz), 6.5-6.6 (m, 1H) ppm.

### (E)-2-Isopropyl-2,4-pentadienoic acid dibutylamide (IID)

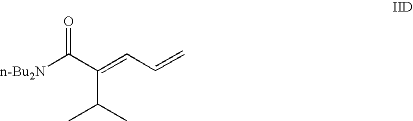

Following the procedure previously described for compound IIC, (E)-2-isopropyl-2,4-pentadienoic acid IIB (1.0 g, 7.18 mmol) can be transformed into amide IID, which can be obtained as a 12:1 E/Z mixture. ¹H NMR (400.13 MHz, CDCl₃) [symbol] 0.7-0.9 (m, 6H), 1.08 (d, 6H, J=7.0 Hz), 1.1-1.3 (m, 4H), 1.3-1.5 (m, 4H), 2.92 (septuplet, 1H, J=7.0 Hz), 3.28 (brs, 2H), 3.19 (brs, 2H), 5.1-5.2 (m, 2H), 5.79 (d, 1H, J=11.0 Hz), 6.5-6.6 (m, 1H) ppm.

### (E)-2-Isopropyl-1-piperidin-1-yl-2,4-pentadien-1-one (IIE)

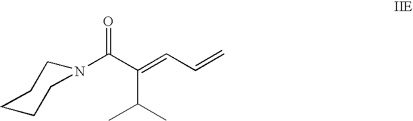

Following the procedure previously described for compound IIC, (E)-2-isopropyl-2,4-pentadienoic acid IIB (660 mg, 4.71 mmol) can be transformed into amide IIE, which can be obtained as a 11:1 E/Z mixture. ¹H NMR (400.13 MHz, CDCl₃) [symbol] 0.9-1.2 (m, 6H), 1.3-1.6 (m, 6H), 2.93 (septuplet, 1H, J=6.9 Hz), 3.38 (brs, 2H), 3.52 (brs, 2H), 5.16 (d, 1H, J=10.0 Hz), 5.19 (d, 1H, J=16.7 Hz), 5.78 (d, 1H, J=11.0 Hz), 6.57 (ddd, 1H, J=16.7, 11.0, 10.0 Hz) ppm.

### (E)-2-Isopropyl-1-trimethylsilanyl-2,4-pentadien-1-one (IIF)

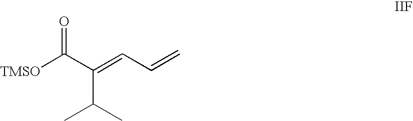

Trimethylsilyl chloride (0.7 mL, 5.5 mmol) is slowly added to a solution of carboxylic acid IIB (700 mg, 5 mmol) and pyridine (0.5 mL, 6 mmol) in CH₂Cl₂ (15 mL) at 0° C. The mixture is then warmed to room temperature and stirred over night. After removing the solvents under reduced pressure, the crude is then dissolved in MTBE (15 mL), filtered and evaporated under vacuum to give (E)-2-isopropyl-1-trimethylsilanyl-2,4-pentadien-1-one IIF. ¹H NMR (400.13 MHz, CDCl₃) δ 0.17 (s, 9H), 1.04 (d, 6H, J=7.0 Hz), 2.89 (septuplet, 1H, J=7.0 Hz), 5.27 (d, 1H, J=10.0 Hz), 5.39 (d, 1H, J=16.7 Hz), 6.71 (ddd, 1H, J=16.7, 11.5, 10.0 Hz), 6.88 (d, 1H, J=11.5 Hz) ppm.

### 2-Isopropyl-2,4-pentadienoic acid 2′-(2-isopropyl-2,4-pentadienoyloxy)biphenyl-2-yl ester (IIaA)

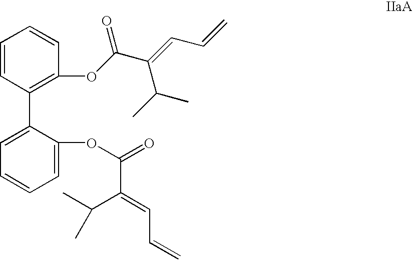

Oxalyl chloride (0.62 mL, 6.6 mmol) is added to a solution of (E)-2-isopropyl-2,4-pentadienoic acid (IIB) (616 mg, 4.4 mmol) in CH₂Cl₂ (5 mL) at 0° C. and the mixture is stirred at room temperature for 1 hour before removing the solvent under reduced pressure. The crude is then dissolved in THF (5 mL) and slowly added to a solution of 2,2′-biphenyldiol (372 mg, 2 mmol) and NaH (176 mg, 60% in oil, 4.4 mmol) in THF (10 mL) at 0° C. that has previously been stirred for 1 hour. After stirring for an extra hour at room temperature, the solution is diluted with EtOAc, washed with saturated aqueous NH₄Cl, water and brine, dried (MgSO₄) and evaporated to give a colorless oil. Purification by column chromatography (SiO₂, 5% EtOAc in hexane) afforded 800 mg of IIaA. ¹H NMR (400.13 MHz, CDCl₃) δ 0.97 (d, 12H, J=7.0 Hz), 2.88 (septuplet, 2H, J=7.0 Hz), 5.34 (d, 2H, J=10.0 Hz), 5.39 (d, 2H, J=16.6 Hz), 6.61 (ddd, 2H, J=16.6, 11.4, 10.0 Hz), 6.84 (d, 2H, J=161.4 Hz), 7.1-7.4 (m, 8H) ppm.

### Diethyl (2E,4E,6E)-2,7-diisopropyl-2,4,6-octatriene-1,8-dioate (IIIA)

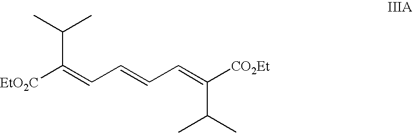

Compound IIA (8.4 g, 50 mmol) is thoroughly deoxygenated by applying vacuum/argon cycles and subsequently warmed up to 40° C. and treated with a solution of Grubbs' second-generation catalyst 2a (21.2 mg, 0.025 mmol, s/c 2000/1) in anhydrous CH₂Cl₂ (5 mL). The mixture is stirred at 40° C. for 4 hours. ¹H NMR analysis of the reaction mixture at this point shows conversion to triene [84% (E,E,E), 6% (E,Z,E), 10% (E,E,Z)]. The mixture is then diluted with MTBE (10 mL), treated with silica gel (5 g), stirred for 15 min and filtered. After removing the solvents under vacuum, the crude is triturated with cold hexane to yield a white solid characterised as IIIA. Alternatively, IIA (8.4 g, 50 mmol) is treated with a solution of Grubbs' second-generation catalyst 2a (42.4 mg, 0.05 mmol, s/c 1000/1) in anhydrous CH₂Cl₂ (10 mL). ¹H NMR analysis of the reaction mixture after 4 hours at 40° C. shows conversion to triene [86% (E,E,E), 6% (E,Z,E), 8% (E,E,Z)]. Compound IIIA is isolated from the reaction crude by trituration with cold hexane. ¹H NMR (400.13 MHz, CDCl₃) δ 1.15 (d, 12H, J=7.0 Hz), 1.24 (t, 6H, J=7.1 Hz), 3.03 (septuplet, 2H, J=7.0 Hz), 4.13 (q, 4H, J=7.1 Hz), 6.81 (m, 2H), 7.06 (m, 2H) ppm.

### (2E,4E,6E)-2,7-Diisopropyl-2,4,6-octatriene-1,8-dioic acid (IIIB)

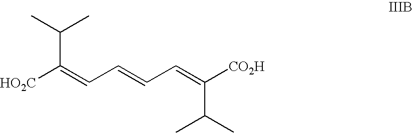

Method 1: A solution of IIIA (7.7 g, 25 mmol) in a 1:1 mixture of THF:MeOH (50 mL) is treated with a 2M aqueous solution of LiOH (37.5 mL, 75 mmol) and stirred over night at 80° C. After cooling down to room temperature the reaction mixture is diluted with water (50 mL) and washed with MTBE. The aqueous phase is acidified by addition of 1M KHSO₄. A white solid precipitates then from the aqueous phase, the solid is filtered, thoroughly washed with water and identified as (2E,4E,6E)-2,7-diisopropyl-2,4,6-octatriene-1,8-dioic acid IIIB. ¹H NMR (400.13 MHz, DMSO) δ 1.21 (d, 12H, J=7.0 Hz), 3.18 (septuplet, 2H, J=7.0 Hz), 7.0-7.2 (m, 4H) ppm.

Method 2: A solution of (IIF) (106 mg, 0.5 mmol) in anhydrous CH₂Cl₂ (0.5 mL) is treated with Grubbs' second-generation catalyst (8.5 mg, 0.01 mmol, 2 mol %) and the mixture is stirred at 40° C. for 24 hours. After cooling down to room temperature, the reaction mixture is diluted with water (1 mL) and washed with MTBE. The aqueous phase is acidified by addition of 1M KHSO₄. A white solid precipitates from the aqueous phase, the solid is filtered, washed thoroughly with water and identified as (2E,4E,6E)-2,7-diisopropyl-2,4,6-octatriene-1,8-dioic acid (IIIB).

Method 3: A solution of IIaA (215 mg, 0.5 mmol) in anhydrous CH₂Cl₂ (100 mL) is treated with Grubbs' second-generation catalyst (21 mg, 0.025 mmol, 5 mol %) and stirred at 40° C. for 24 hours. The solution is then stirred with silica gel (100 mg) for 15 min and filtered. After removing the solvents under vacuum the crude is dissolved in a 1:1 mixture of THF:MeOH (1 mL), treated with a 2M aqueous solution of LiOH (1 mL, 2 mmol) and stirred over night at 80° C. After cooling down to room temperature the reaction mixture is diluted with water (5 mL) and washed with MTBE. The aqueous phase is acidified by addition of 1M KHSO₄. A white solid precipitates then from the aqueous phase, the solid is filtered, thoroughly washed with water and identified as 3:1 E,E,E)/(E,Z,E) octatrienedioic acid IIIB.

### (2E,4E,6E)-2,7-Diisopropyl-2,4,6-octatriene-1,8-dioic acid bisdiisopropyl amide (IIIC)

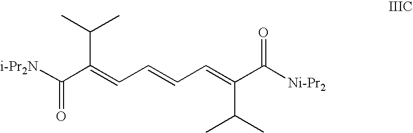

A solution of IIC (1.4 g, 6.1 mmol) in anhydrous CH₂Cl₂ (18 mL) is treated with Grubbs' second-generation catalyst (105 mg, 0.122 mmol, 2 mol %) and the mixture is stirred at 40° C. for 24 hours. The solution is then treated with silica gel (2.0 g), stirred for 15 min and filtered. After removing the solvents under vacuum, compound IIIC can be obtained as a 3:1 E,E,E/E,Z,E mixture. Compound IIIC is then dissolved in hexane (50 mL), treated with a small crystal of iodine and stirred at room temperature for 48 h. The solution is then washed with 0.19 M aqueous sodium thiosulphate (30 mL), dried (MgSO₄) and evaporated to give IIIC as a 11:1 E,E,E/E,Z,E mixture. ¹H NMR (400.13 MHz, CDCl₃) δ0.9-1.3 (m, 24H), 1.39 (brs, 12H), 2.91 (septuplet, 2H, J=7.0 Hz), 3.32 (brs, 2H), 4.07 (brs, 2H), 5.8-5.9 (m, 2H), 6.4-6.5 (m, 2H) ppm.

### (S)-4-Benzyl-3-[(S)-2-isopropyl-4-pentenoyl)-2-oxazolidinone (IVA)

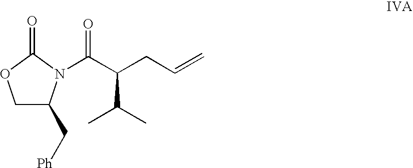

To a stirred solution of (S)-4-benzyl-3-(3-methylbutyryl)-2-oxazolidinone (13.0 g, 50 mmol), which is prepared according to Rueger et al Tetrahedron Letters (2000), 41(51), 10085-10089, in dry THF at −78° C. is added LiHMDS (55 mL, 1.0 M in toluene, 55 mmol) and the solution is stirred at 0° C. for 30 minutes before cooling down to −78° C. Allyl bromide (4.0 mL, 55 mmol) is then added and the mixture is stirred at room temperature for 2 hours. The products are extracted with EtOAc, washed with saturated aqueous NH₄Cl, water and saturated aqueous NaCl, dried (MgSO₄) and evaporated to give a yellow oil which is purified by flash chromatography on silica gel eluting with 10% EtOAc/hexane to give IVA as a colourless oil. ¹H NMR (400.13 MHz, CDCl₃) δ 0.91 (d, 6H, J=6.8 Hz), 1.8-2.0 (m, 1H), 2.2-2.5 (m, 2H), 2.57 (dd, 1H, J=13.3, 10.1 Hz), 3.25 (dd, 1H, J=13.3, 3.2 Hz), 3.7-3.9 (m, 1H), 4.0-4.1 (m, 2H), 4.5-4.7 (m, 1H), 4.95 (d, 1H, J=10.2 Hz), 5.02 (dq, 1H, J=17.1, 1.5 Hz), 5.7-5.8 (m, 1H), 7.1-7.3 (m, 5H) ppm.

### (S)-2-Isopropyl-4-pentenoic acid (IVB)

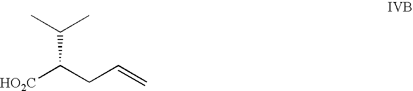

To a stirred solution of IVA (19.0 g, 63.1 mmol) in THF (135 mL) and water (35 mL) at 0° C. is added H₂O₂ (37 mL, 35% w/v in water, 366 mmol) rapidly followed by aqueous LiOH (70 mL, 2.6 M in water, 183 mmol). After stirring for 1 hour at 0° C. the solution is warmed to room temperature and stirred overnight. Aqueous Na₂SO₃ (70 mL, 0.5 M in water, 35 mmol) is then added followed by water (70 mL) and the aqueous phase is washed with MTBE (2×100 mL, MTBE washings may be evaporated to recover the cleaved chiral auxiliary). The aqueous phase is then made acidic (pH=1) on addition of 10% aqueous HCl and products are extracted with MTBE. The organic phase is washed with water and saturated NaCl, dried (MgSO₄) and evaporated (250 mbar at 40° C.) to give IVB as a light yellow oil containing MTBE. This MTBE solution is used directly in the next step. ¹H NMR (400.13 MHz, CDCl₃) δ 0.82 (d, 3H, J=6.8 Hz), 0.83 (d, 3H, J=6.8 Hz), 1.7-1.9 (m, 1H), 2.0-2.3 (m, 3H), 4.87 (d, 1H, J=10.2 Hz), 4.93 (dq, 1H, J=17.1, 1.6 Hz), 5.62 (ddt, 1H, J=17.1, 10.2, 6.8 Hz) ppm.

### Methyl (S)-2-isopropyl-4-pentenoate (IVC)

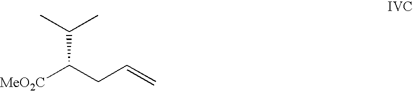

To a solution of IVB (2.5 g, 17.6 mmol) in acetone (50 mL) is added MeI (3.3 mL, 52.8 mmol) and K₂CO₃ (3.66 g, 26.4 mmol) and the mixture is stirred at room temperature overnight. The solution is then evaporated (250 mbar at 40° C.) diluted with MTBE, washed with water a saturated aqueous NaCl, dried (MgSO₄) and evaporated (250 mbar at 40° C.) to give IVC as a colourless oil. ¹H NMR (400.13 MHz, CDCl₃) δ 0.84 (d, 3H, J=6.8 Hz, CHCH₃), 0.88 (d, 3H, J=6.8 Hz, CHCH₃), 1.8-1.9 (m, 1H), 2.0-2.3 (m, 3H), 3.59 (s, 3H), 4.90 (d, 1H, J=10.2 Hz), 4.93 (dq, 11-1, J=17.1, 1.6 Hz), 5.66 (ddt, 1H, J=17.1, 10.2, 6.8 Hz) ppm.

### (S)-2-Isopropylpent-4-enoyl chloride (IVD)

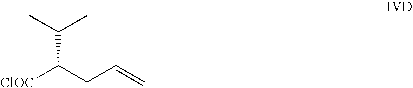

A solution of IVB (2.11 g) in 22 ml CH₂Cl₂ is treated with 1-chloro-N,N-2-trimethylpropenylamine (2.95 mL). After stirring for 5 h at RT the solution is concentrated and used for the next step without further purification.

(S)-2-Isopropylpent-4-enoic acid 2-((S)-2-isopropylpent-4-enoyloxymethyl)benzyl ester (IVaA)

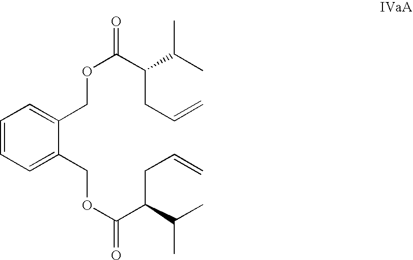

To a solution of pyridine (1 ml) in CH₂Cl₂ (3.5 mL) is added a solution of 1,2 benzenedimethanol (250 mg, 1.76 mmol) in 5 ml CH₂Cl₂ at 0° C. After 20 min. a solution of IVD (crude 2.75 g [7 mmol] in 5 ml DCM) and DMAP (38 mg) are added and the solution is stirred for 16 h at RT. The mixture is diluted with EtOAc (10 ml) and HCl (5 mL, 1N) is added. The organic phase is separated from the water phase, dried over Na₂SO₄ and evaporated to give IVaA. Purification by flash chromatography (EtOAc/hexanes 1:15 to 1:5) gives a colourless oil. ¹H NMR (400.13 MHz, CDCl₃) δ 0.90 (d, J=6.8 Hz, 6H), 0.94 (d, J=6.8 Hz, 6H), 1.90 (m, 2H), 2.22-2.40 (m, 6H), 4.93-5.05 (m, 4H), 5.20 (s, 4H), 5.65-5.80 (m, 2H), 7.32-7.45 (m, 4H). MS (M+NH4)=405.

### (S)-2-Isopropylpent-4-enoic acid 3-((S)-2-isopropylpent-4-enoyloxymethyl)benzyl ester (IVaB)

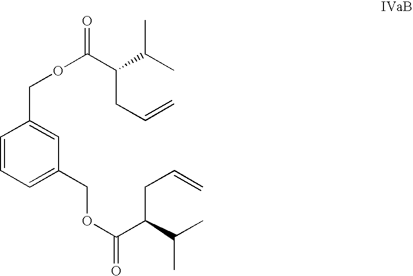

To a solution of pyridine (0.5 ml) in CH₂Cl₂ (5 mL) is added 1,3 benzenedimethanol (194 mg, 1.41 mmol) at 0° C. After 20 min. a solution of IVD (crude 677 mg [4 mmol] in 5 ml of CH₂Cl₂) and DMAP (20 mg) are added. The solution is stirred for 16 h at RT. The mixture is diluted with EtOAc (10 ml) and HCl (5 mL, 1N) is added. The organic phase is separated from the water phase, dried over Na₂SO₄ and evaporated to give IVaB. Purification by flash chromatography (EtOAc/hexanes 1:15 to 1:5) gives a colourless oil. ¹H NMR (400.13 MHz, CDCl₃) δ 0.91 (d, J=7.1 Hz, 6H), 0.94 (d, J=7.1 Hz, 6H), 1.90 (m, 2H), 2.22-2.40 (m, 6H), 4.93-5.07 (m, 4H), 5.10 (s, 4H), 5.65-5.80 (m, 2H), 7.28-7.40 (m, 4H).

### (S)-2-Isopropylpent-4-enoic acid 2-((R)-2-isopropylpent-4-enoyloxy)phenyl ester (IVaC)

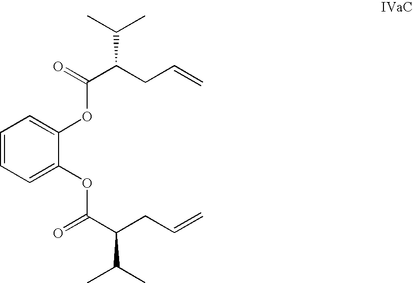

To a solution of pyridine (2.8 ml) in CH₂Cl₂ (8 mL) is added a solution of benzene-1,2-diol (472 mg, 4.3 mmol) in CH₂Cl₂ (36 mL) at 0° C. After 20 min, a solution of IVD (crude 2 g [12.9 mmol] in 8 ml of CH₂Cl₂) is added and the solution is stirred for 3 h at 0-5° C. HCl (25 mL, 1N) is added. The organic phase is separated from the water phase and dried over Na₂SO₄ and evaporated. Purification by flash chromatography (EtOAc/hexanes 1:15 to 1:5) gives IVaC as a colourless oil. ¹H NMR (400.13 MHz, CDCl₃) δ 1.05 (dd, J=6.5 Hz, 12H), 2.1 (m, 2H), 2.30-2.55 (m, 6H), 5.13 (d, J=24.8 2H), 5.18 (d, J=28.2, 2H), 5.88 (m, 2H), 7.20 (m, 4H). MS (M+NH4)=376.

### (8S,13S)-8,13-Diisopropyl-5,8,9,12,13,16-hexahydro-6,15-dioxa-benzocyclotetradecene-7,14-dione (IcA)

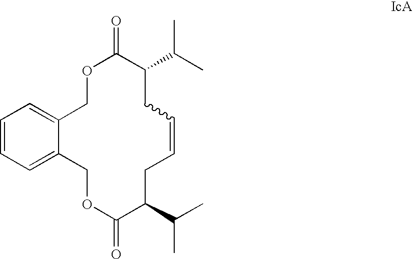

A solution of IVaA (80 mg, 0.2 mmol) in anhydrous CH₂Cl₂ (2 mL) is treated with Grubbs' second-generation catalyst 2a (10.5 mg, 0.012 mmol, s/c 100/6) and the mixture is stirred at room temperature for 24 hours. The solution is then treated with silica gel (1.0 g), stirred for 15 min and filtered. After flash chromatography (EtOAc/hexanes 1:15 to 1:5), compound IcA is obtained as a solid in a 10:1 E:Z ratio. (E)-IcA: ¹H NMR (400.13 MHz, CDCl₃) δ 0.91 (d, J=6.7 Hz, 6H), 0.94 (d, J=6.9 Hz, 6H), 1.81 (m, 2H), 2.10-2.30 (m, 6H), 4.93 (d, J=12.3 Hz, 2H), 5.44 (d, J=12.7 Hz, 2H), 5.46 (s, 2H), 5.65-5.80 (m, 2H), 7.28-7.37 (m, 4H).

### (5S,10S)-5,10-Diisopropyl-3,12-dioxabicyclo[12.3.1]octadeca-1(17), 7.14 (18), 15-tetraene-4,1′-dione (IcB)

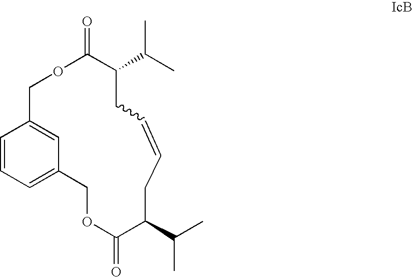

A solution of IVaB (94 mg, 0.24 mmol) in anhydrous CH₂Cl₂ (2 mL) is treated with Grubbs' second-generation catalyst 2a (12 mg, 0.015 mmol, s/c 100/6) and the mixture is stirred at room temperature for 15 hours. The solution is then treated with silica gel (1.0 g), stirred for 15 min and filtered. After flash chromatography (EtOAc/hexanes 1:15 to 1:5), compound IcB is obtained as an oil in 10:1 E:Z ratio. (E)-IcB: ¹H NMR (400.13 MHz, CDCl₃) δ 0.95 (d, J=6.7 Hz, 12H), 1.80-1.96 (m, 2H), 2.10-2.40 (m, 6H), 5.04 (d, J=12.7 Hz, 2H), 5.34 (d, J=12.2 Hz, 2H), 5.30 (s, 2H), 7.17-7.40 (m, 4H). MS (M+NH4)=376.

### (7S,12R)-7,12-Diisopropyl-7,8,11,12-tetrahydro-5,14-dioxa-benzocyclo dodecene-6,13-dione (IcC)

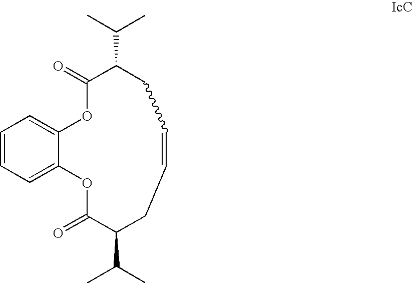

A solution of IVaC (100 mg, 0.28 mmol) in anhydrous toluene (2.8 mL) is treated with Grubbs' second-generation catalyst 2a (0.48 mg, 0.0006 mmol, s/c 500/1) and the mixture is stirred at 50° C. for 5 hours. The solution is then treated with silica gel (1.0 g), stirred for 15 min and filtered. After flash chromatography (EtOAc/hexanes 1:15 to 1:5), compound IcC is obtained as a solid in 10:1 E:Z ratio. (E)-IcC: ¹H NMR (400.13 MHz, CDCl₃) δ 1.03 (d, J=6.7 Hz, 6H), 1.04 (d, J=6.6, 6H), 1.87-1.97 (m, 2H), 2.13-2.20 (m, 2H), 2.40-2.55 (m, 4H), 5.58 (m, 2H), 5.88 (m, 2H), 7.05 (m, 2H), 7.25 (m, 2H).

### Dimethyl (2S,7S)-(E)-2,7-diisopropyl-4-octene-1,8-dioate (IA)

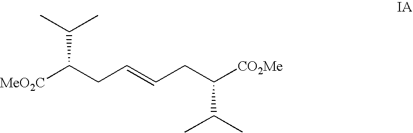

A solution of IVC (312 mg, 2.0 mmol) in anhydrous CH₂Cl₂ (6 mL) is treated with Grubbs' second-generation catalyst 2a (17 mg, 0.02 mmol, s/c 100/1) and the mixture is stirred at 40° C. for 24 hours. The solution is then treated with silica gel (1.0 g), stirred for 15 min and filtered. After removing the solvents under vacuum, compound IA is obtained as a 5:1 E/Z mixture (as determined by GC analysis). ¹H NMR (400.13 MHz, CDCl₃) δ 0.89 (d, 6H, J=6.7 Hz), 0.92 (d, 6H, J=6.7 Hz), 1.7-1.9 (m, 2H), 2.1-2.3 (m, 6H), 3.65 (s, 6H), 5.37 (s, 2H) ppm. GC analysis: Chiraldex G-PN, 10 psi, 150-200° C. over 23 min, retention times: Z-IA 17.18 min, E-IA 17.76 min.

### (2S,7S)-(E)-2,7-Diisopropyl-4-octene-1,8-dioic acid (IB)

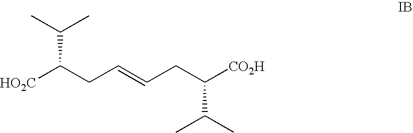

A solution of a 5:1 E/Z mixture of IA (256 mg, 0.9 mmol) in a 1:1 mixture THF:MeOH (1.8 mL) is treated with a 2M aqueous solution of LiOH (1.8 mL, 3.6 mmol) and the mixture is stirred over night at 80° C. After cooling down to room temperature the reaction mixture is acidified by careful addition of 1M KHSO₄ and extracted with MTBE (3×). The combined organic phases are dried (MgSO₄) and evaporated to give a 5:1 E/Z mixture of IB as a white solid. ¹H NMR (400.13 MHz, CDCl₃) δ 0.87 (dd, 12H, J=6.5, 2.1 Hz), 1.76 (m, 2H), 2.0-2.2 (m, 6H), 5.33 (s, 2H) ppm.

### (E)-2,7-Diisopropyl-4-octene-1,8-dioic acid (IB)

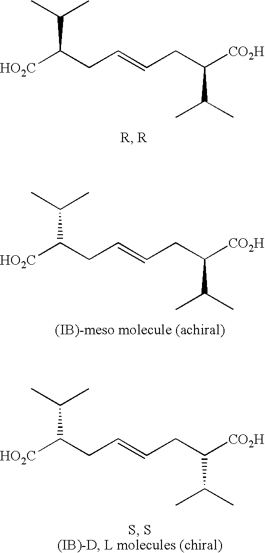

In a 25 mL glass-liner is added [(R)-phenethyl-(R)-BoPhozRuCl (benzene)]Cl (1.1 mg, 0.001 mmol, s/c 1000/1). This is placed in the Parr autoclave and the air replaced with hydrogen. A solution of IIIB (252 mg, 1 mmol) and Et₃N (0.26 mL, 2 mmol) in methanol (5 mL) is then added to the Parr autoclave. The autoclave is then pressurised with hydrogen to 10 bar and left to stir at room temperature. After 1 hour the uptake of hydrogen is stopped. The autoclave is opened and the solution analysed by ¹H NMR. NMR analysis shows a 7:1 (IB)-D,L to (IB)-meso ratio (according to integration of vinylic proton signals at 5.33 and 5.37 ppm, respectively).

The separation of (IB)-D,L and (IB)-meso cab be achieved, for example, via recrystallization of diastereomeric salts by several procedures well known to persons skilled in the art (e.g. Kozma, D. CRC Handbook of Optical Resolutions via Diastereomeric Salt Formation, CRC Press, 2002). For example (IB)-(S,S) can be separated via salt formation with (S)-phenylethylamine.

### 1,8-Bis-((S)-4-benzyl-2-oxo-oxazolidin-3-yl)-2,7-diisopropyl-4-octene-1,8-dione (IC)

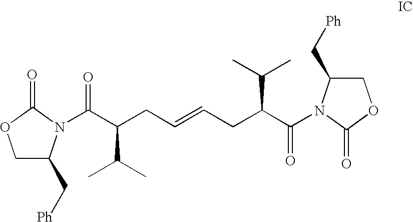

A solution of IVA (100 mg, 0.33 mmol) in methylenechloride (4 mL) is treated with Grubbs' second-generation catalyst (14 mg, 0.016 mmol, s/c 100/5) and the mixture is stirred at 50° C. for 18 hours. The solution is then treated with silica gel (1.0 g), stirred for 15 min and filtered. After flash chromatography (EtOAc/hexanes 1:15 to 1:5) compound IC is obtained as solid in 9:1 E:Z ratio. (E)-IC: ¹H NMR (400.13 MHz, CDCl₃): δ 0.86 (t, J=7.0 Hz, 12H), 1.90 (m, 2H), 2.18-2.40 (m, 4H), 2.61 (d, J=13.3 Hz, 1H), 2.63 (d, J=13.3 Hz, 1H), 3.27 (dd, J=3.2, 13.1 Hz, 2H), 3.67-3.75 (m, 2H), 4.03-4.08 (m, 4H), 4.55-4.65 (m, 2H), 5.46 (m, 2H), 5.88 (m, 2H), 7.13-7.30 (m, 10H).

### Preparation of Catalyst 3

A solution of N-Di(3,5-difluorophenyl)phosphine N-methyl S-1-(R-2-diphenylphosphino) ferrocenylethylamine (0.1 g, 0.146 mmol) and [RuCl₂(benzene)]₂ (0.036 g, 0.073 mmol) in ethanol (2 ml) and toluene (1 ml) was stirred under a N₂ atmosphere at 60° C. for 15 min. The solvent was removed in vacuo and the solid redissolved in dichloromethane (1 ml). Methyl tent-butyl ether (5 ml) was added, which resulted in precipitation of an orange solid. This solid was collected by filtration and dried to give catalyst 3 as an orange solid. ³¹P NMR (162 MHz, CDCl₃) δ 85 (d) and 19 (d) ppm.

### Preparation of Catalyst 8

A solution of N-Diphenylphosphine N—(R)-phenylethenyl R-1-(S-2-diphenylphosphino) ferrocenylethylamine (0.035 g, 0.05 mmol) and [RuCl₂(benzene)]₂ (0.0125 g, 0.005 mmol) in ethanol (1 ml) and toluene (0.5 ml) was stirred under a N₂ atmosphere at 60° C. for 60 min. The solvent was removed in vacuo and the solid redissolved in dichloromethane (1 ml). Methyl tert-butyl ether (5 ml) was added, which resulted in precipitation of an orange solid. This solid was collected by filtration and dried to give catalyst 8 as an orange solid. ³¹P NMR (162 MHz, CDCl₃) δ 78 (d) and 21 (d) ppm.

### Preparation of Catalysts 1, 2, 4, 5, 6, 7 and 9

Catalysts 1, 2, 4, 5, 6, 7 and 9 are prepared following analogous procedures to those described above for 3 and 8. The corresponding ligands for the preparation of these catalysts are: N-diphenylphosphine N-methyl S-1-(R-2-diphenylphosphino)ferrocenylethylamine (1 and 9), N-di(4-fluorophenyl)phosphine N-methyl S-1-(R-2-diphenylphosphino) ferrocenylethylamine (2), N—(R)-BINOL-phosphinite N-methyl R-1-(S-2-diphenylphosphino)ferrocenylethylamine (4), N—(S)-BINOL-phosphinite N-methyl R-1-(S-2-diphenylphosphino) ferrocenylethylamine (5), N-di(4-trifluoromethylphenyl)phosphine N-methyl S-1-(R-2-diphenylphosphino) ferrocenylethylamine (6) and N-diphenylphosphine N-benzyl R-1-(S-2-diphenylphosphino) ferrocenylethylamine (7).

³¹P NMR (162 MHz, CDCl₃) δ 84 (d) and 22 (d) ppm for [(S)-BoPhoz RuCl (benzene)]Cl R⁸=Me, R⁹=phenyl (catalyst 1);

³¹P NMR (162 MHz, CDCl₃) δ 85 (d) and 22 (d) ppm for [(S)-BoPhoz RuCl (benzene)]Cl R⁹=Me, R⁹=p-fluorophenyl (catalyst 2);

³¹P NMR (162 MHz, CDCl₃) δ 143 (d) and 28 (d) ppm for [(R)-BoPhoz RuCl (benzene)]Cl R⁸=Me, R⁹═(R)-binol (catalyst 4);

³¹P NMR (162 MHz, CDCl₃) δ 148 (d) and 33 (d) ppm for [(R)-BoPhoz RuCl (benzene)]Cl R⁸=Me, R⁹═(S)-binol (catalyst 5);

³¹P NMR (162 MHz, CDCl₃) δ 85 (d) and 20 (d) ppm for [(S)-BoPhoz RuCl (benzene)]Cl R⁸=Me, R⁹=p-CF₃-phenyl (catalyst 6);

³¹P NMR (162 MHz, CDCl₃) δ 114 (d) and 43 (d) for [(S)-BoPhoz RuCl (benzene)]Cl R⁸=Me, R⁹=Benzyl (catalyst 7).

Catalyst 9 is prepared in situ and used directly without characterisation.

For preparation procedures of ligands see: Boaz, N. W.; Ponasik, J. A. Jr.; Large, S. E.; Tetrahedron: Asymmetry 2005, 16, 2063; Boaz, N. W.; Mackenzie, E. B.; Debenham, S. D.; Large, S. E.; Ponasik, J. A. Jr. J. Org. Chem. 2005, 70, 1872; Li, X.; Jia, X.; Xu, L.; Kok, S. H. L.; Yip, C. W.; Chan, A. S. C. Adv. Synth. Catal. 2005, 347, 1904 and Boaz, N. W.; Ponasik, J. A., Jr.; Large, S. E. Tetrahedron Lett. 2006, 47, 4033. For preparation of ligands in catalysts 4 and 5 see also Jia, X.; Li, X.; Lam, W. S.; Kok, S. H. L.; Xu, L.; Lu, G.; Yeung, C.-H.; Chan, A. S. C. Tetrahedron: Asymmetry 2004, 15, 2273.

### Salt Formation of (S,S)-diisopropyl-oct-4-enedioic acid with (S)-phenylethylamine

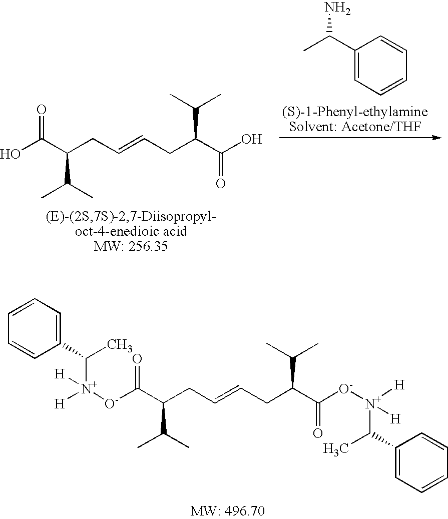

2 g (6.6 mmol) of crude diacid are dissolved in 5 ml of acetone at room temperature. Then 0.8 g (6.6 mmol, 1 equiv) of (S)-phenylethylamine is added and the yellow solution is stirred for 30 min at room temperature. Another equiv of (S)-phenylethylamine (0.8 g, 6.6 mmol) is added. After 30 min, a thick crystalline suspension is formed. 3 ml of THF is added and stirring is continued at 0° C. for 30 min. A first crop is isolated by filtration and dried to give bis-(S)-phenylethylamine salt. From the mother liquor is isolated a second crop by addition of heptane. 0.15 g of the first crop is recrystallized from 1 ml DCM and 1 ml THF. After standing over night, white crystals are isolated and dried (mp. 136-138° C.)

¹H-NMR: (400 MHz,), δ_{H} (ppm) 0.70-0.85 (12H, 2d, overlap, —CH₃), 1.2-1.3 (2H, brm, —CH), 1.4-1.55 (2H, brm, —CH), 1.6-1.7 (6H, d, 2×CH₃), 1.80-1.95 (4H, brm, allyl-CH), 4.18-4.25 (2H, q, —CH₃), 4.8-4.9, 2H, m, olef.-H), 7.25-7.4 (6H, brm, arom.-H), 7.5-7.6 (4H, d, o-arom.—H), 8.2-9.8 (6H, very br., 2×—NH₃⁺).

Esterification of Diacid with 2 Equiv Iodomethane to Dimethylester

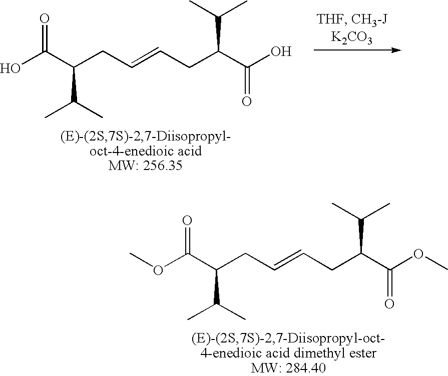

6.0 g (23.4 mmol) of optically pure E-(2S,7S)-diisopropyl-oct-4-endioic acid, which is prepared by dissolving the (S)-phenylethylamine salt from the previous experiment in water, acidifying to pH 2 and extracting the acid with EtOAc and concentrating the organic phase, are dissolved in 50 ml of N-methylpyrrolidone. 12 ml of water is added, followed by the addition of 10.0 g of potassium carbonate (72.5 mmol) to give a slightly turbid solution. Under stirring, 9.97 g (70.2 mmol) of methyl iodide is added via a dropping funnel. The temperature is raised to 40° C. and stirring is continued overnight. After complete conversion (20 h). The crude reaction mixture is partitioned between 80 ml of water and 50 ml of TBME. The organic phase is extracted several times with 50 ml portions of TBME and then the combined organic phase is washed with 3×50 ml of water. The organic phase is evaporated under vacuum and next degassed in high vacuum for 30 min. to give the desired diester product.

¹H-NMR: (400 MHz, CDCl3), δ_{H} (ppm) 0.8-0.85 (6H, d, 2×-CH₃), 0.85-0.90 (6H, d, 2×-CH₃), 1.7-1.83 (2H, oct., —CH), 2.05-2.22 (6H, brm, allyl-H & —COOR), 3.60 (6H, s, —OCH₃), 5.28-5.35 (2H, m, olef.—H).

[α]_{D}=−6.3 (1% in MeOH); [α]_{D}=−8.1 (1% in Dichloromethane)

### Bromohydrine Formation

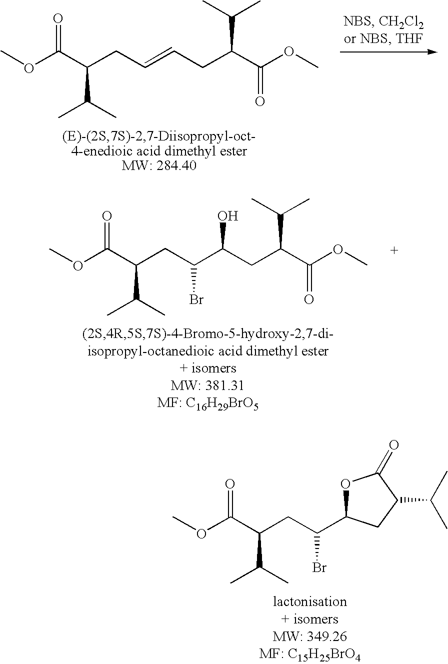

5.4 g (18.98 mmol) of (S,S)-diisopropyl octenedioic diester from the previous experiment is dissolved in 33 ml of THF, followed by the addition of 26 ml of water. To the biphasic emulsion is added in 2 portions (3.76 g, 20.8 mmol) of N-bromosuccinimide. The mixture is stirred at room temperature for 1 hour. HPLC control shows complete conversion of the starting material (to give a mixture of two products in the ratio of 92:8; area-%). To the reaction mixture is added 25 ml of TBME to separate the phases. The aqueous phase is extracted twice with 25 ml of TBME. The combined organic phases are washed with water and are then dried over MgSO₄. The organic phase is evaporated under vacuum to give a yellow oil. After the workup procedure, HPLC shows a changed product mixture (30:70). NMR and LC-MS shows that the major product after the workup and thermal treatment is the desired bromolactone methylester and the minor product consists of the bromohydrine dimethylester.

HPLC retention times: olefin diester, 11.05 min; bromolactone monoester, 10.17 min. and bromohydrine diester, 9.70 min.

HPLC column: Inertsil ODS-3V (C-18, 5m), 4.6 mm×250 mm; 40° C.; flow: 1.5 ml/min. Solvent system: water (0.01 NH₄H₂PO₄): acetonitrile, gradient 45:55 to 3:97

IR: (FTIR-microscopy in transmission, in [cm⁻¹] of “bromohydrine diester” (contaminated with little lactone): 3501 (—OH), 2963 (as, CCH₃), 2876 (s, CCH₃), 1780 (lactone, weak), 1732 (ester, strong), 1466, 1437, 1373, 1244, 1201, 1160

LC-MS: M⁺=381.31 (corresponds to C₁₆H₂₉O₅Br)

M⁺=349.10 (corresponds to C₁₅H₂₅O₄Br)

### Lactonization to Bromolactone

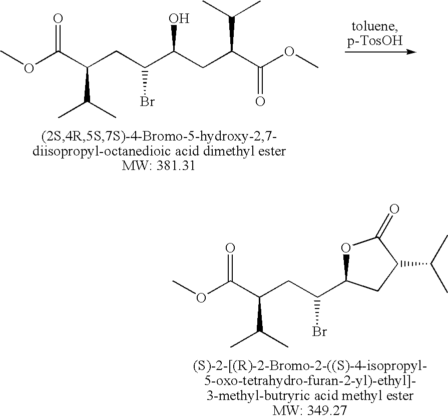

The residue of the previous experiment, which is a mixture of bromohydrine diester and bromolactone monoester (7.1 g), and 380 mg of p-TosOH is dissolved in 40 ml of toluene and heated to reflux for 7 hours to complete lactonisation. After aqueous workup and evaporation the desired product is obtained, which shows, according to NMR analysis, two diastereomeric components in the ratio 20:80.

¹H-NMR: (400 MHz, CDCl₃), δ_{H} (ppm) 0.9-1.10 (12H, overlap d, —CH₃), 1.72-1.82 (1H, m), 1.85-1.95 (1H, m), 1.95-2.05 (1H, m), 2.15-2.30 (2H, brm), 2.35-2.50 (2H, brm), 2.60-2.70 (2H, brm), 3.70 (3H, s, —OCH₃), 3.95-4.10 (1H, brm, 2 brm, ratio (4:1)), 4.30-4.50 (1H, brm, 2 brm, ratio (4:1).

IR: (FTIR-microscopy in transmission, in [cm⁻¹] of “bromolactone monoester”; 2963, 2876, 1779 (lactone), 1732 (ester), 1467, 1437, 1372, 1199, 1161

Displacement with Sodium Azide in DMF to Azidolactone Methylester

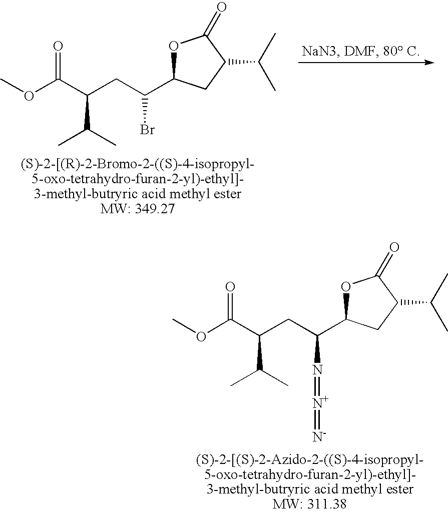

1.5 g (4.3 mmol) of bromolactone monoester diastereomer mixture from the previous experiment are dissolved in 10 ml of DMF. 0.83 g of NaN₃ (12.76 mmol) are added and the mixture is heated up to 70° C. for 12 hours. The mixture is then cooled down to room temperature and then diluted with 20 ml of water. The product is isolated by several extractions between water and TBME. Drying the combined organic phases over MgSO₄ and evaporation gives the azidolactone monoester as a mixture of diastereomers.

MS: LC-MS: M+NH₄⁺=329, three different isomers

IR: FTIR-microscopy in transmission, in [cm⁻¹]; 2963, 2876, 2110 (—N₃), 1782 (lactone), 1733 (ester), 1700 (side prod.), 1468, 1437, 1373, 1264, 1195, 1161, 1119

### Hydrogenation of Azido-Lactone Methylester to Lactam-Lactone

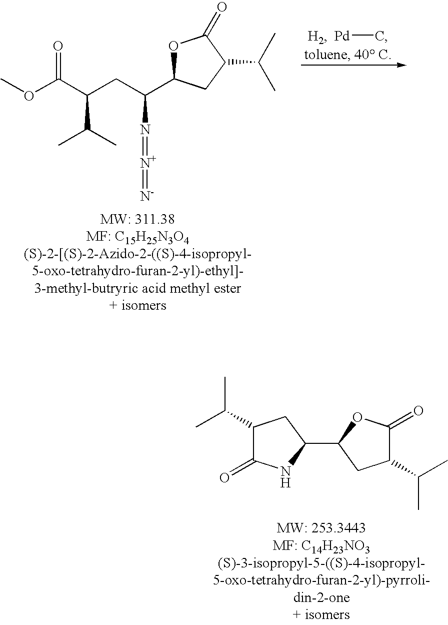

1.5 g of azido-lactone methylester (4.8 mmol) are dissolved in 15 ml of toluene. 0.5 g of Pd/C (5%) catalyst (Engelhard 4522) are added and hydrogenation is performed at room temperature under 1 atm pressure over 24 hours. The catalyst is filtered and the filtrate is evaporated in vacuum to give a semi crystalline off white material, which contains according to ¹H-NMR, IR, HPLC and TLC the desired (S,S,S,S) compound along with two other diastereomeric lactam-lactone compounds.

¹H-NMR (400 MHz, CDCl₃): δ=6.04 (s, 1H), 4.22-4.16 (m, 1H), 3.51-3.46 (m, 1H), 2.55-2.51 (m, 1H), 2.44-2.38 (m, 1H), 2.17-2.09 (m, 3H), 2.07-1.99 (m, 1H), 1.94-1.87 (m, 1H), 1.80-1.73 (m, 1H) 0.99-0.97 (d, 3H), 0.95-0.93 (d, 3H), 0.91-0.89 (d, 3H), 0.85-0.84 (d, 3H)

IR: 1776=lactone, 1704=lactam, cm⁻¹ (FTIR-Microscopy in transmission)

1. A process for preparing a compound of formula (I) 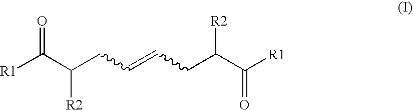
wherein
R1 is OR3 or NR4R5;
R2 is C₁₋₇alkyl or C₃₋₈cycloalkyl;
R3 is hydrogen, C₁₋₇alkyl, phenyl- or naphthyl-C₁₋₄alkyl, aryl, heterocyclyl or C₃₋₈cycloalkyl, each unsubstituted or substituted: or is SiRR′R″, wherein R, R′ and R″ are independently of each other C₁₋₇alkyl, aryl or phenyl-C₁₋₄alkyl:
R4 and R5 are independently hydrogen, C₁₋₇alkyl, phenyl- or naphthyl-C₁₋₄alkyl, aryl, heterocyclyl or C₃₋₈cycloalkyl, each unsubstituted or substituted;
or R4 and R5 may form together a 3 to 7 membered nitrogen containing saturated hydrocarbon ring, which may contain one or more heteroatoms selected from N or O and, which can be unsubstituted or substituted:
or a salt thereof;
said process comprising one or more of the following steps:
a) subjecting a compound of formula (II), or a salt thereof, 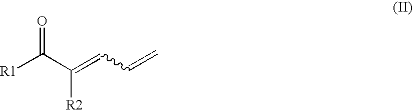
wherein R1 and R2 are as defined for a compound of formula (I), to cross-metathesis reaction to obtain a compound of formula (III), or a salt thereof, 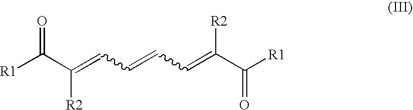
wherein R1 and R2 are as defined for a compound of formula (I);
b) subjecting said compound of formula (III), or a salt thereof, to hydrogenation to obtain a compound of formula (I), or a salt thereof.

2. A process for preparing a compound of formula (III) 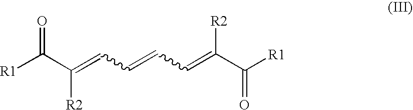
wherein
R1 is OR3 or NR4R5;
R2 is C₁₋₇alkyl or C₃₋₈cycloalkyl:
R3 is hydrogen, C₁₋₇alkyl, phenyl- or naphthyl-C₁₋₄alkyl, aryl, heterocyclyl or C₃₋₈cycloalkyl, each unsubstituted or substituted; or is SiRR′R″, wherein R, R′ and R″ are independently of each other C₁₋₇alkyl, aryl or phenyl-C₁₋₄alkyl;
R4 and R5 are independently hydrogen, C₁₋₇alkyl, phenyl- or naphthyl-C₁₋₄alkyl, aryl,
heterocyclyl or C₃₋₈cycloalkyl, each unsubstituted or substituted;
or R4 and R5 may form together a 3 to 7 membered nitrogen containing saturated hydrocarbon ring, which may contain one or more heteroatoms selected from N or O and, which can be unsubstituted or substituted;
or a salt thereof;
said process comprising the step of subjecting a compound of formula (II), or a salt thereof, 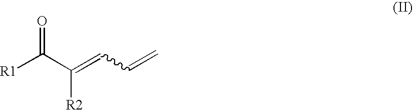
wherein R1 and R2 are as defined for a compound of formula (III), to cross-metathesis reaction to obtain a compound of formula (III), or a salt thereof.

3. A process for preparing a compound of formula (I) 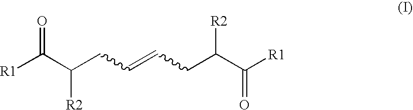
wherein
R1 is OR3 or NR4R5;
R2 is C₁₋₇alkyl or C₃₋₈cycloalkyl;
R3 is hydrogen, C₁₋₇alkyl, phenyl- or naphthyl-C₁₋₄alkyl, aryl, heterocyclyl or C₃₋₈cycloalkyl, each unsubstituted or substituted; or is SiRR′R″, wherein R, R′ and R″ are independently of each other C₁₋₇alkyl, aryl or phenyl-C₁₋₄alkyl;
R4 and R5 are independently hydrogen, C₁₋₇alkyl, phenyl- or naphthyl-C₁₋₄alkyl, aryl, heterocyclyl or C₃₋₈cycloalkyl, each unsubstituted or substituted;
or R4 and R5 may form together a 3 to 7 membered nitrogen containing saturated hydrocarbon ring, which may contain one or more heteroatoms selected from N or O and, which can be unsubstituted or substituted;
or a salt thereof;
said process comprising the step of subjecting a compound of formula (III), or a salt thereof, 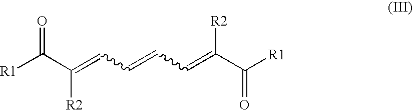
wherein R1 and R2 are as defined for a compound of formula (I), to hydrogenation to obtain a compound of formula (I), or a salt thereof.

4. A process for the preparation of a renin inhibitor comprising one or more of the following steps:
a. subjecting a compound of formula (II), or a salt thereof, 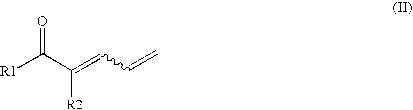
wherein R1 and R2 are as defined for a compound of formula (I), to cross-metathesis reaction to obtain a compound of formula (III), or a salt thereof, 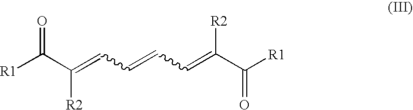
wherein R1 and R2 are as defined for a compound of formula (I);
b. subjecting a compound of formula (III), or a salt thereof, wherein R1 and R2 are as defined for a compound of formula (I), to hydrogenation to obtain a compound of formula (I) 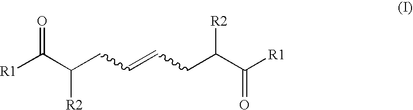
wherein
R1 is OR3 or NR4R5;
R2 is C₁₋₇alkyl or C₃₋₈cycloalkyl;
R3 is hydrogen, C₁₋₇alkyl, phenyl- or naphthyl-C₁₋₄alkyl, aryl, heterocyclyl or C₃₋₈cycloalkyl, each unsubstituted or substituted; or is SiRR′R″, wherein R, R′ and R″ are independently of each other C₁₋₇alkyl, aryl or phenyl-C₁₋₄alkyl;
R4 and R5 are independently hydrogen, C₁₋₇alkyl, phenyl- or naphthyl-C₁₋₄alkyl, aryl, heterocyclyl or C₃₋₈cycloalkyl, each unsubstituted or substituted;
or R4 and R5 may form together a 3 to 7 membered nitrogen containing saturated hydrocarbon ring, which may contain one or more heteroatoms selected from N or O and, which can be unsubstituted or substituted;
or a salt thereof.

5. A process for preparing a compound of formula (I) 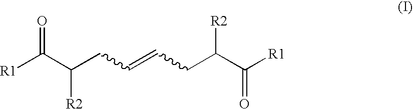
wherein
R1 is OR3 or NR4R5;
R2 is C₁₋₇alkyl or C₃₋₈cycloalkyl;
R3 is hydrogen. C₁₋₇alkyl, phenyl- or naphthyl-C₁₋₄alkyl, aryl, heterocyclyl or C₃₋₈cycloalkyl, each unsubstituted or substituted; or is SiRR′R″, wherein R, R′ and R″ are independently of each other C₁₋₇alkyl, aryl or phenyl-C₁₋₄alkyl;
R4 and R5 are independently hydrogen, C₁₋₇alkyl, phenyl- or naphthyl-C₁₋₄alkyl, aryl, heterocyclyl or C₃₋₈cycloalkyl, each unsubstituted or substituted;
or R4 and R5 may form together a 3 to 7 membered nitrogen containing saturated hydrocarbon ring, which may contain one or more heteroatoms selected from N or O and, which can be unsubstituted or substituted;
or a salt thereof:
said process comprising one or more of the following steps:
a) subjecting a compound of formula (IIa), or a salt thereof, 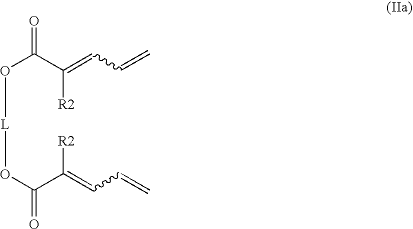
wherein
L is a linker connecting the two oxygen atoms via a 1 to 6 carbon backbone and
R2 is as defined for a compound of formula (I) to cross-metathesis reaction to obtain a compound of formula (IIIb), or a salt thereof, 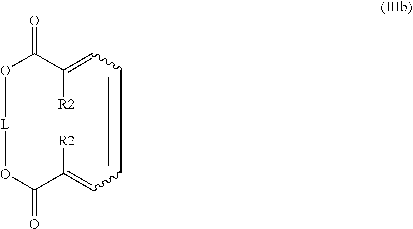
wherein L and R2 are as defined for said compound of formula (IIa):
b) converting said compound of formula (IIIb), or a salt thereof, into a compound of formula (I), or a salt thereof, by either submitting said compound of formula (IIIb), or a salt thereof, to hydrogenation followed by hydrolysis or to hydrolysis followed by hydrogenation.

6. A process for preparing a compound of formula (IIIb) 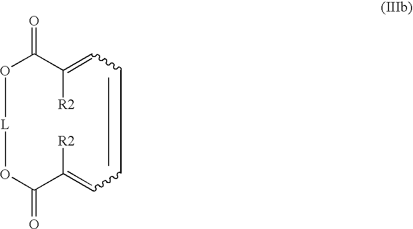
wherein
L is a linker connecting the two oxygen atoms via a 1 to 6 carbon backbone and
R2 is C₁₋₇alkyl or C₃₋₈cycloalkyl;
or a salt thereof,
said process comprising the step of subjecting a compound of formula (IIa), or a salt thereof, 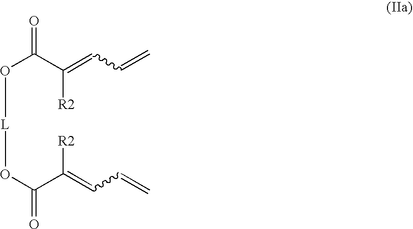
wherein
L and R2 are as defined for a compound of formula (IIIb), to cross-metathesis reaction to obtain a compound of formula (IIIb), or a salt thereof.

7. A process for preparing a compound of formula (I) 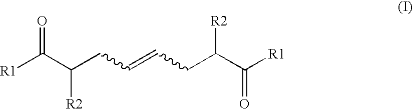
wherein
R1 is OR3 or NR4R5;
R2 is C₁₋₇alkyl or C₃₋₈cycloalkyl;
R3 is hydrogen, C₁₋₇alkyl, phenyl- or naphthyl-C₁₋₄alkyl, aryl, heterocyclyl or C₃₋₈cycloalkyl, each unsubstituted or substituted: or is SiRR′R″, wherein R, R′ and R″ are independently of each other C₁₋₇alkyl, aryl or phenyl-C₁₋₄alkyl;
R4 and R5 are independently hydrogen, C₁₋₇alkyl, phenyl- or naphthyl-C₁₋₄alkyl, aryl, heterocyclyl or C₃₋₈cycloalkyl, each unsubstituted or substituted;
or R4 and R5 may form together a 3 to 7 membered nitrogen containing saturated hydrocarbon ring, which may contain one or more heteroatoms selected from N or O and, which can be unsubstituted or substituted;
or a salt thereof;
said process comprising the step of converting a compound of formula (IIIb), or a salt thereof, 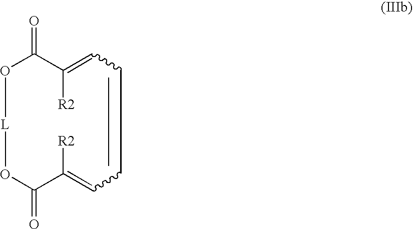
wherein
L is a linker connecting the two oxygen atoms via a 1 to 6 carbon backbone and
R2 is as defined for a compound of formula (I), into a compound of formula (I), or a salt thereof, by either submitting said compound of formula (IIIb), or a salt thereof, to hydrogenation followed by hydrolysis or to hydrolysis followed by hydrogenation.

8. A process for the preparation of a renin inhibitor comprising one or more of the following steps:
a subjecting a compound of formula (IIa), or a salt thereof, 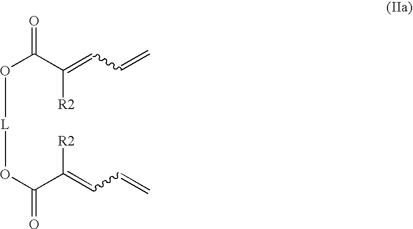
wherein
L is a linker connecting the two oxygen atoms via a 1 to 6 carbon backbone and
R2 is as defined for a compound of formula (I), to cross-metathesis reaction to obtain a compound of formula (IIIb), or a salt thereof, 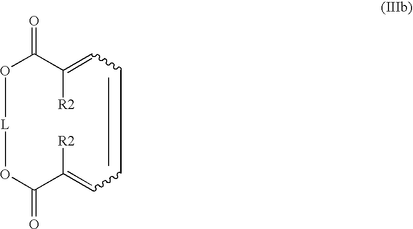
wherein L and R2 are as defined for said compound of formula (IIa);
c. converting said compound of formula (IIIb), or a salt thereof, into a compound of formula (I), or a salt thereof, by either submitting said compound of formula (IIIb), or a salt thereof, to hydrogenation followed by hydrolysis or to hydrolysis followed by hydrogenation.

9. A process for preparing a compound of formula (I) 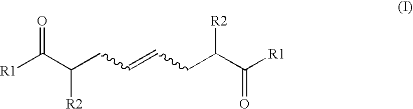
wherein
R1 is OR3 or NR4R5;
R2 is C₁₋₇alkyl or C₃₋₈cycloalkyl:
R3 is hydrogen, C₁₋₇alkyl, phenyl- or naphthyl-C₁₋₄alkyl, aryl, heterocyclyl or C₃₋₈cycloalkyl, each unsubstituted or substituted; or is SiRR′R″, wherein R, R′ and R″ are independently of each other C₁₋₇alkyl, aryl or phenyl-C₁₋₄alkyl;
R4 and R5 are independently hydrogen, C₁₋₇alkyl, phenyl- or naphthyl-C₁₋₄alkyl, aryl, heterocyclyl or C₃₋₈cycloalkyl, each unsubstituted or substituted;
or R4 and R5 may form together a 3 to 7 membered nitrogen containing saturated hydrocarbon ring, which may contain one or more heteroatoms selected from N or O and, which can be unsubstituted or substituted:
or a salt thereof;
said process comprising subjecting a compound of formula (IV), or a salt thereof, 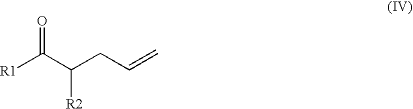
wherein R1 and R2 are as defined for a compound of formula (I), to cross-metathesis reaction to obtain a compound of formula (I), or a salt thereof.

10. A process for the preparation of a renin inhibitor comprising subjecting a compound of formula (IV), or a salt thereof, 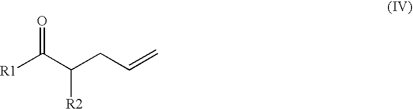
wherein R1 and R2 are as defined for a compound of formula (I), to cross-metathesis reaction to obtain a compound of formula (I) 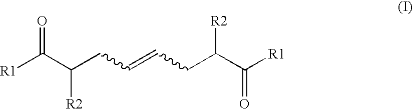
wherein
R1 is OR3 or NR4R5;
R2 is C₁₋₄alkyl or C₃₋₈cycloalkyl;
R3 is hydrogen, C₁₋₇alkyl, phenyl- or naphthyl-C₁₋₄alkyl, aryl, heterocyclyl or C₃₋₈cycloalkyl, each unsubstituted or substituted: or is SiRR′R″, wherein R, R′ and R″ are independently of each other C₁₋₇alkyl, aryl or phenyl-C₁₋₄alkyl;
R4 and R5 are independently hydrogen, C₁₋₇alkyl, phenyl- or naphthyl-C₁₋₄alkyl, aryl, heterocyclyl or C₃₋₈cycloalkyl, each unsubstituted or substituted;
or R4 and R5 may form together a 3 to 7 membered nitrogen containing saturated hydrocarbon ring, which may contain one or more heteroatoms selected from N or O and, which can be unsubstituted or substituted;
or a salt thereof;

11. A process for preparing a compound of formula (I) 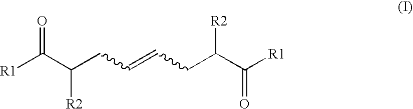
wherein
R1 is OR3 or NR4R5:
R2 is C₁₋₇alkyl or C₃₋₈cycloalkyl;
R3 is hydrogen, C₁₋₇alkyl, phenyl- or naphthyl-C₁₋₄alkyl, aryl, heterocyclyl or C₃₋₈cycloalkyl, each unsubstituted or substituted; or is SiRR′R″, wherein R, R′ and R″ are independently of each other C₁₋₇alkyl, aryl or phenyl-C₁₋₄alkyl;
R4 and R5 are independently hydrogen, C₁₋₇alkyl, phenyl- or naphthyl-C₁₋₄alkyl, aryl, heterocyclyl or C₃₋₈cycloalkyl, each unsubstituted or substituted:
or R4 and R5 may form together a 3 to 7 membered nitrogen containing saturated hydrocarbon ring, which may contain one or more heteroatoms selected from N or O and, which can be unsubstituted or substituted;
or a salt thereof;
said process comprising one or more of the following steps:
a) subjecting a compound of formula (IVa), or a salt thereof, 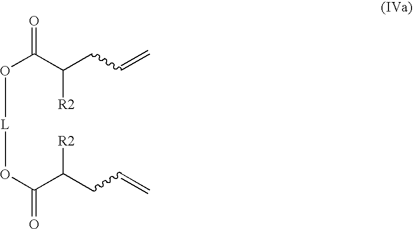
wherein
L is a linker connecting the two oxygen atoms via a 1 to 6 carbon backbone and
R2 is as defined for a compound of formula (I), to cross-metathesis reaction to obtain a compound of formula (Ic), or a salt thereof, 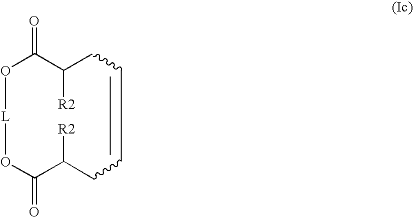
wherein L and R2 are as defined for said compound of formula (IVa);
b) converting said compound of formula (Ic), or a salt thereof, into a compound of formula (I), or a salt thereof, by hydrolysis reaction.

12. A process for the preparation of a renin inhibitor comprising one or more of the following steps:
a subjecting a compound of formula (IVa), or a salt thereof, 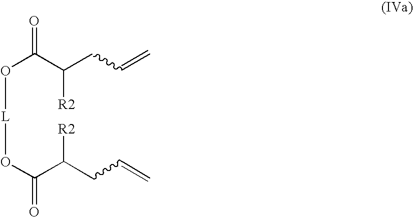
wherein
L is a linker connecting the two oxygen atoms via a 1 to 6 carbon backbone and
R2 is as defined for a compound of formula (I), to cross-metathesis reaction to obtain a compound of formula (Ic), or a salt thereof, 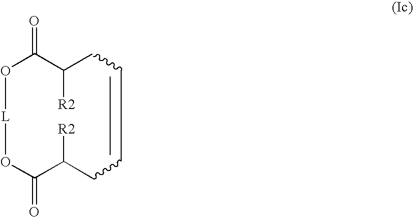
wherein L and R2 are as defined for said compound of formula (IVa);
b converting said compound of formula (Ic), or a salt thereof, into a compound of formula (I), or a salt thereof, by hydrolysis reaction.

13. The process according to any one of claim 1, 3, 5, 7, 9, or 11 wherein the compound of formula (I), or a salt thereof, has a structure according to formula (Ia) 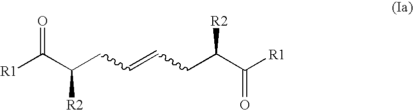
wherein R1 and R2 are as defined for a compound of formula (I).

14. The process according to any one of claim 1, 3, 5, 7, 9 or 11 wherein the compound of formula (I), or a salt thereof, has a structure according to formula (Ib) 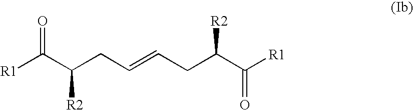
wherein R1 and R2 are as defined for a compound of formula (I).

15. The process according to any one of claim 4, 8, 10 or 12 wherein the rennin inhibitor is aliskiren.

16. The process according to any one of claim 1, 2, 4 to 6, 8 or 12 wherein the cross-metathesis reaction employs a ruthenium alkylidene catalyst.

17. The process according to claim 16 wherein the ruthenium alkylidene catalyst is selected from the group consisting of:

| | |
|---|---|
| 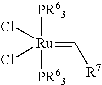 | 1a, R⁶ = Cyclohexenyl, R⁷ = Ph 1b, R⁶ = Cyclohexenyl, R⁷ = CH₂Ph 1c, R⁶ = ⁱPr, R⁷ = C₅H₁₁ 1d, R⁶ = ⁱPr, R⁷ = C₇H₁₅ 1e, R⁶ = ⁱPr, R⁷ = CH₂Ph 1f, R⁶ = ⁱPr, R⁷ = CH₂SPh 1g, R⁶ = ⁱPr, R⁷ = CHCPh₂ |
| 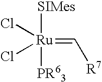 | 2a, R⁶ = Cyclohexenyl, R⁷ = Ph 2b, R⁶ = ⁱPr, R⁷ = CH₂Ph 2c, R⁶ = ⁱPr, R⁷ = CH₂SPh 2d, R⁶ = Ph, R⁷ = CH₂Ph 2e, R⁶ = Tol, R⁷ = CH₂Ph 2f, R⁶ = p-MeOC₆H₄, R⁷ = CH₂Ph 2g, R⁶ = C₇H₁₅, R⁷ = ⁱPr |
| 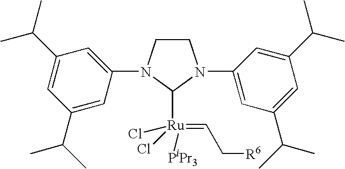 | 3a, R⁶ = C₄H₉ 3b, R⁶ = C₆H₁₃ 3c, R⁶ = Ph |
| 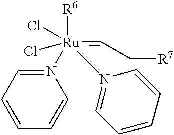 | 4a, R⁶ = IMes, R⁷ = Ph 4b, R⁶ = SIMes, R⁷ = Ph 4c, R⁶ = SIMes, R⁷ = C₆H₁₃ |
| 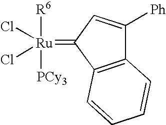 | 5a, R⁶ = SIMes 5b, R⁶ = P(Cyclohexenyl)₃ |
| 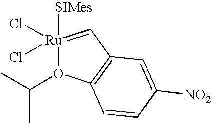 | 6a |
| 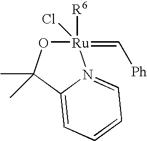 | 7a, R⁶ = P(Cyclohexenyl)₃ 7b, R⁶ = SIMes 7c, R⁶ = P(ⁱPr)₃ |
| 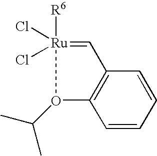 | 8a, R⁶ = P(Cyclohexenyl)₃ 8b, R⁶ = SIMes |
| 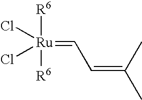 | 9a, R⁶ = P(Cyclohexenyl)₃ |
| 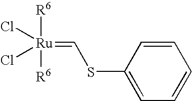 | 10a, R⁶ = P(Cyclohexenyl)₃ |

18. The process according to any one of claim 1, 3 to 5, 7 or 8 wherein the hydrogenation reaction employs a ruthenium catalyst.

19. The process according to claim 18 wherein the ruthenium catalyst is selected from the group consisting of: 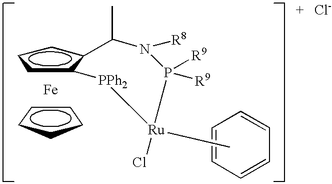
1-9

| | | |
|---|---|---|
| 1 | [(S)-BoPhoz RuCl benzene)]Cl | R⁸ = Me, R⁹ = Ph |
| 2 | [(S)-BoPhoz RuCl (benzene)]Cl | R⁸ = Me, R⁹ = p-fluorophenyl |
| 3 | [(S)-BoPhoz RuCl (benzene)]Cl | R⁸ = Me, |
| | | R⁹ = 3,5-difluorophenyl |
| 4 | [(R)-BoPhoz RuCl (benzene)]Cl | R⁸ = Me, R⁹ = (R)-binol |
| 5 | [(R)-BoPhoz RuCl (benzene)]Cl | R⁸ = Me, R⁹ = (S)-binol |
| 6 | [(S)-BoPhoz RuCl (benzene)]Cl | R⁸ = Me, R⁹ = p-CF₃phenyl |
| 7 | [(R)-BoPhoz RuCl (benzene)]Cl | R⁸ = Bn, R⁹ = Ph |
| 8 | [(R)-BoPhoz RuCl (benzene)]Cl | R⁸ = (R)-phenethyl, R⁹ = Ph |
| 9 | (S)-BoPhoz RuCl₂ dmf | R⁸ = Me, R⁹ = Ph |

20. A compound of formula (III) 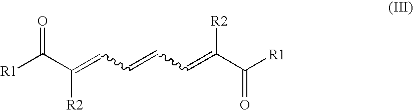
wherein
R1 is OR3 or NR4R5;
R2 is branched C₁₋₇alkyl or C₃₋₈cycloalkyl:
R3 is hydrogen, C₁₋₇alkyl, phenyl- or naphthyl-C₁₋₄alkyl, wherein phenyl- or naphthyl are unsubstituted or substituted, unsubstituted or substituted aryl, unsubstituted or substituted heterocyclyl or unsubstituted or substituted C₃₋₈cycloalkyl; or is SiRR′R″, wherein R, R′ and R″ are independently of each other C₁₋₇alkyl, aryl or phenyl-C₁₋₄alkyl;
R4 and R5 are independently hydrogen, C₁₋₇alkyl, phenyl- or naphthyl-C₁₋₄alkyl, wherein phenyl- or naphthyl are unsubstituted or substituted, unsubstituted or substituted aryl, unsubstituted or substituted heterocyclyl or unsubstituted or substituted C₃₋₈cycloalkyl;
or R4 and R5 may form together a 3 to 7 membered nitrogen containing saturated hydrocarbon ring, which may contain one or more heteroatoms selected from N or O and, which can be unsubstituted or substituted:
or a salt thereof.

21. A compound according to claim 20 having the following structure 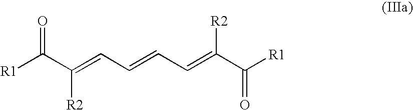
: or a salt thereof.

22. A compound according to claim 21 wherein
R1 is OH and R2 is a branched C₁₋₇alkyl

23. A compound of formula (I) 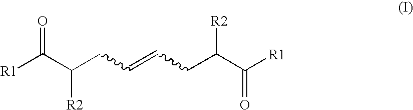
wherein
R1 is OR3 or NR4R5;
R2 is branched C₁₋₇alkyl or C₃₋₈cycloalkyl;
R3 is hydrogen, C₁₋₇alkyl, phenyl- or naphthyl-C₁₋₄alkyl, wherein phenyl- or naphthyl- is unsubstituted or substituted, unsubstituted or substituted aryl, unsubstituted or substituted heterocyclyl or unsubstituted or substituted C₃₋₈cycloalkyl; or is SiRR′R″, wherein R, R′ and R″ are independently of each other C₁₋₇alkyl, aryl or phenyl-C₁₋₄alkyl;
R4 and R5 are independently hydrogen, C₁₋₇alkyl, phenyl- or naphthyl-C₁₋₄alkyl, wherein phenyl- or naphthyl- is unsubstituted or substituted, unsubstituted or substituted aryl, unsubstituted or substituted heterocyclyl or unsubstituted or substituted C₃₋₈cycloalkyl;
or R4 and R5 may form together a 3 to 7 membered nitrogen containing saturated hydrocarbon ring, which may contain one or more heteroatoms selected from N or O and, which can be unsubstituted or substituted by one or more, e.g. one to four substitutents for example independently selected from the group of hydroxyl, halo, oxo, amino, alkylamino, dialkylamino, thiol, alkylthio, nitro, hydroxy-C₁-C₇-alkyl, halo-C₁-C₇-alkyl, C₁-C₇-alkyl, C₁-C₇alkanoyl, such as acetyl, C₁-C₇alkoxy, halo-C₁-C₇-alkoxy, such as trifluoromethoxy, hydroxy-C₁-C₇alkoxy, C₁-C₇-alkoxy-C₁-C₇-alkoxy, carbamoyl, cyano and aryl-C₁-C₇-alkyl, wherein aryl is substituted;
or a salt thereof.

24. A compound according to claim 23 having the following structure 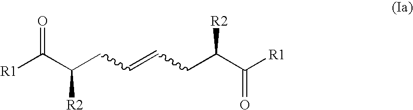
: or a salt thereof.

25. A compound of formula (Ib), or a salt thereof, 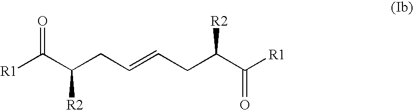
wherein
R1 is OH and
R2 is a branched C₁₋₇ alkyl.

26. The compound of formula 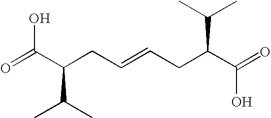
or salt thereof.

27. A compound of formula (IIa), or a salt thereof, 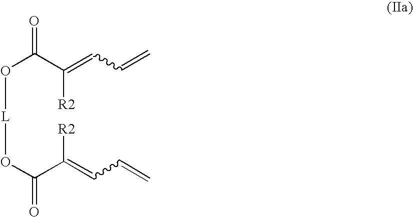
wherein
L is a linker connecting the two oxygen atoms via a 1 to 6 carbon backbone and
R2 is a branched C₁₋₇ alkyl.

28. A compound of formula (IIIb), or a salt thereof, 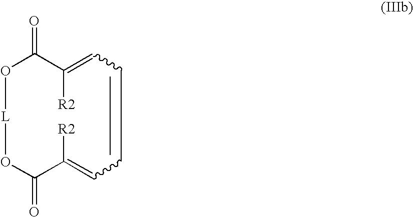
wherein
L is a linker connecting the two oxygen atoms via a 1 to 6 carbon backbone and
R2 is a branched C₁₋₇ alkyl.

29. A compound of formula (IVa), or a salt thereof, 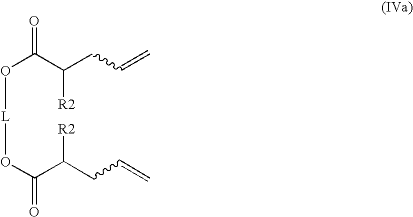
wherein
L is a linker connecting the two oxygen atoms via a 1 to 6 carbon backbone and
R2 is a branched C₁₋₇ alkyl.

30. A compound of formula (Ic), or a salt thereof, 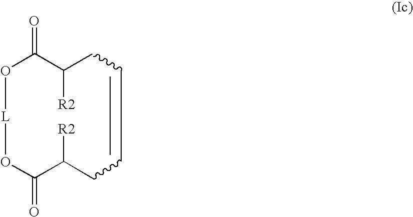
wherein
L is a linker connecting the two oxygen atoms via a 1 to 6 carbon backbone and
R2 is a branched C₁₋₇ alkyl.

31-32. (canceled)
